(19) United States
(12) Patent Application Publication
(10) Pub. No.: US 2026/0315552 A1
(43) Pub. Date: Oct. 15, 2026

(54) COMBINATION OF VITAMINS, 2'-FUCOSYLLACTOSE AND A PROBIOTIC FOR ENHANCING BONE GROWTH AND/OR BONE STRENGTH

(71) Applicant: SOCIÉTÉ DES PRODUITS NESTLÉ S.A., Vevey (CH)

(72) Inventors: Esther CAMPOS GIMENEZ, Billens (CH); Marie Noëlle HORCAJADA, Echenevex (FR); Nicolas BONNET, Beaumont (FR)

(21) Appl. No.: 19/485,406

(22) PCT Filed: May 24, 2024
(86) PCT No.: PCT/EP2024/064341

§ 371 Date: Nov. 18, 2025

(30) Foreign Application Priority Data
May 25, 2023 (EP) 23175290.8

(51) Int. Cl. A61K 35/745; A61K 31/07; A61K 31/122; A61K 31/592; A61K 31/593; A61K 31/702; A61K 35/747; A61P 19/00

(52) U.S. Cl. CPC A61K 35/745; A61K 31/07; A61K 31/122; A61K 31/592; A61K 31/593; A61K 31/702; A61K 35/747; A61P 19/00

(57) ABSTRACT

The present invention provides a combination of a vitamin mixture and a synbiotic for use in enhancing bone growth and/or bone strength, for example in a child who has suffered from and/or is suffering from stunted growth and/or faltering growth.

### FIELD OF THE INVENTION

The present invention relates to compositions and methods for enhancing bone growth and/or bone strength, for example in a child who has suffered from and/or is suffering from stunted growth and/or faltering growth.

### BACKGROUND TO THE INVENTION

The growth and development of the human skeleton requires an adequate supply of many different nutritional factors. Classical nutrient deficiencies are associated with stunting (e.g. energy, protein, Zn), rickets (e.g. vitamin D) and other bone abnormalities (e.g. Cu, Zn, vitamin C). There is evidence to suggest that peak bone mass and later fracture risk are influenced by the pattern of growth and nutritional exposures in childhood. However, it is challenging to define dietary reference values using bone health as a criterion, and the question of what type of diet constitutes the best support for optimal bone growth and development remains open (see e.g. Prentice, A., et al., 2006. Proceedings of the Nutrition Society, 65 (4), pp. 348-360).

Several approaches may be taken to improve the intake of growth-limiting nutrients, including administration of micronutrient supplements, fortification of food with micronutrients or improved dietary intake. However, particularly in populations where dietary quality is poor, several micronutrient deficiencies can co-occur, in which case growth may be affected by more than one growth-limiting nutrient (see e.g. Rivera, J. A., et al., 2003. The Journal of nutrition, 133 (11), pp. 4010S-4020S).

Thus, there is a demand for new nutritional interventions to enhance bone growth and/or bone strength, for example in children suffering from stunted growth and/or faltering growth.

### SUMMARY OF THE INVENTION

The present inventors have surprisingly found that a combination of vitamin K2, vitamin A, and vitamin D synergistically promotes osteoblast mineralization. Further, the present inventors have surprisingly found that a combination of vitamins K2, A, and D with short chain fatty acids (SCFAs) synergistically promotes osteoblast differentiation. SCFAs may be produced, for example, by oligosaccharide fermentation in the gut by probiotic bacteria.

In one aspect, the present invention provides a combination of a vitamin mixture and a synbiotic for use in enhancing bone growth and/or bone strength in a subject, wherein the vitamin mixture comprises or consists of vitamin K2, vitamin A, and vitamin D.

In another aspect, the present invention provides use of a combination of a vitamin mixture and a synbiotic in the manufacture of a medical food product for enhancing bone growth and/or bone strength in a subject, wherein the vitamin mixture comprises or consists of vitamin K2, vitamin A, and vitamin D.

In another aspect, the present invention provides a method for enhancing bone growth and/or bone strength in a subject, the method comprising administering the subject an effective amount of a combination of a vitamin mixture and a synbiotic, wherein the vitamin mixture comprises or consists of vitamin K2, vitamin A, and vitamin D.

The subject may be any suitable subject, in particular the subject may be a child or a juvenile. The subject may be a human child. For example, the subject may be a human about 3 years of age or older and/or about 10 years of age or younger. In some embodiments, the subject is a human from about 3 years to about 10 years of age. Alternatively, the subject may be a juvenile animal, for example a juvenile pet. The subject may have suffered from and/or may be suffering from stunted growth and/or faltering growth. The subject may have been born preterm, with low-birth weight, and/or experienced intra-uterine growth retardation.

The synbiotic may be any suitable mixture of prebiotics and probiotics. For example, the synbiotic may comprise 2′-fucosyllactose (2′FL) and one or more probiotic. For example, the synbiotic may comprise 2′-fucosyllactose (2′FL) and Bifidobacterium infantis.

The synbiotic may comprise an oligosaccharide mixture. The oligosaccharide mixture may be any suitable mixture of two or more oligosaccharides. For example, the oligosaccharide mixture may comprise or consist of 2′-fucosyllactose (2′FL) and at least one further oligosaccharide. Suitably, the at least one further oligosaccharide comprises or consists of at least one sialylated oligosaccharide, at least one fucosylated oligosaccharide, and/or at least one N-acetylated oligosaccharide. Suitably, the at least one sialylated oligosaccharide is selected from the group consisting of 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof. In some embodiments, the at least one sialylated oligosaccharide is selected from 3′-sialyllactose (3′-SL), 6′-sialyllactose (6′-SL) and combinations thereof. In some embodiments, the at least one sialylated oligosaccharide is 6′-sialyllactose (6′-SL). Suitably, the at least one fucosylated oligosaccharide is selected from the group consisting of 3-fucosyllactose (3FL), difucosyllactose (diFL), lacto-N-fucopentaose-I (LNFP-I), lacto-N-fucopentaose-II (LNFP-II), lacto-N-fucopentaose-III (LNFP-III), lacto-N-fucopentaose-V (LNFP-V), lacto-neofucopentaose V (LNnFP-V), lacto-N-difucosylhexaose-I (LNDFH-1), lacto-N-neodifucosylhexaose (LNnDFH), monofucosyllacto-n-hexaose-III (MFNLH-III), difucosyllacto-N-hexaose-a (DFLNHa) and combinations thereof. In some embodiments, the at least one fucosylated oligosaccharide is difucosyllactose (diFL). Suitably, the at least one N-acetylated oligosaccharide is selected from the group consisting of N-acetyl-glucosamine, N-acetyl-galactosamines, lacto-N-tetraose (LNT), lacto-N-neotetraose (LNnT), and combinations thereof. In some embodiments, the at least one N-acetylated oligosaccharide is selected from lacto-N-tetraose (LNT), lacto-N-neotetraose (LNnT) and combinations thereof. In some embodiments, the at least one N-acetylated oligosaccharide is lacto-N-tetraose (LNT) and lacto-N-neotetraose (LNnT). Suitably, the oligosaccharide mixture comprises: (a) about 10 to about 35 wt %, about 10 to about 30 wt %, or about 10 to about 25 wt %, with respect to the total weight of the oligosaccharide mixture, of at least one sialylated oligosaccharide; (b) about 30 to about 80 wt %, about 40 to about 80 wt %, or about 50 to about 70 wt %, with respect to the total weight of the oligosaccharide mixture, of at least one fucosylated oligosaccharide; and/or (c) about 10 to about 35 wt %, about 15 to about 30 wt %, or about 15 to about 20 wt %, with respect to the total weight of the oligosaccharide mixture, of at least one N-acetylated oligosaccharide.

The synbiotic may comprise any suitable probiotic. Suitably, the one or more probiotic comprises or consists of Bifidobacterium infantis. In some embodiments, the synbiotic comprises a probiotic mixture comprising B. infantis and at least one further probiotic. Suitably, the at least one further probiotic comprises or consists of Bifidobacterium animalis and/or Lactobacillus rhamnosus.

The combination may be administered by any suitable route. Suitably, the combination is administered by oral administration. The combination may be administered separately, simultaneously or sequentially. In some embodiments, the combination is administered simultaneously.

The combination may be administered to the subject in any suitable amounts. Suitably, vitamin K2 is administered to the subject in an amount of from about 5 μg/day to about 200 μg/day. Suitably, vitamin A is administered to the subject in an amount of from about 100 μg RE/day to about 1000 μg RE/day. Suitably, vitamin D is administered to the subject in an amount of from about 2.5 μg/day to about 100 μg/day. Suitably, 2′FL is administered to the subject in an amount of from about 0.5 g/day to about 5 g/day. Suitably, B. infantis is administered to the subject in a total amount of from about 10⁶ cfu/day to about 10¹² cfu/day.

The combination may be provided in any suitable form, for example in the form of a composition. The combination may be provided in the form of a nutritional composition. The combination may be provided in the form of a medical food product for clinical nutrition. The combination may be provided in the form of a milk formula.

The composition may comprise the combination in any suitable amounts. Suitably, the composition comprises vitamin K2 in an amount of from about 5 μg/100 g to about 200 μg/100 g, on a dry weight basis. Suitably, the composition comprises vitamin A in an amount of from about 100 μg RE/100 g to about 1000 μg RE/100 g, on a dry weight basis. Suitably, the composition comprises vitamin D in an amount of from about 2.5 μg/100 g to about 20 μg/100 g, or from about 5 μg/100 g to about 20 μg/100 g on a dry weight basis. Suitably, the composition comprises 2′FL in an amount of from about 0.5 wt % to about 5 wt %, on a dry weight basis. Suitably, the composition comprises B. infantis in an amount of from about 10⁶ cfu/100 g to about 10¹² cfu/100 g, on a dry weight basis.

The combination may synergistically enhance bone growth and/or bone strength. The combination may enhance bone mineralization. The combination may promote osteoblast mineralization and/or osteoblast differentiation. The combination may promote catch-up growth. Suitably, catch-up growth is determined using height velocity.

The present inventors have also surprisingly found that vitamin K2 production in the gastrointestinal tract (e.g. via conversion of vitamin K1) is promoted by a synbiotic.

In another aspect, the present invention provides use of a synbiotic to promote vitamin K2 production in the gut of a subject. The synbiotic may be any described herein.

In another aspect, the present invention provides a method for promoting vitamin K2 production in the gut of a subject, the method comprising administering to the subject an effective amount of a synbiotic. The synbiotic may be any described herein.

### DESCRIPTION OF DRAWINGS

FIG. 1—Effect of vitamins K2, A, D on osteoblast alkaline phosphatase (ALP) activity and osteocalcin mRNA levels in a pre-osteoblastic cell line

(A) ALP activity after 7 days of differentiation (without ascorbic acid). (B) Osteocalcin mRNA levels after 21 days of differentiation (without ascorbic acid). (C) Osteocalcin mRNA levels after 28 days of differentiation (with ascorbic acid). Pos=positive control; Neg=negative control; MK-7=supplemented with 3 μM menaquinone-7; VitD=supplemented with 1 nM 1α,25-Dihydroxyvitamin D3; VitA=supplemented with 100 nM all-trans-retinoic acid.

FIG. 2—Effect of vitamins K2, A, D and short chain fatty acids (SCFA) on osteoblast ALP activity in a pre-osteoblastic cell line

ALP activity after 7 days of differentiation (without ascorbic acid). Pos=positive control; Neg=negative control; MK-7+A+D=supplemented with 3 μM menaquinone-7, 1 nM 1α,25-Dihydroxyvitamin D3, and 100 nM all-trans-retinoic acid; SCFA 20 μM=supplemented with 15 μM of sodium acetate, 4M of sodium propionate and 1 μM of sodium butyrate; SCFA 50 μM=supplemented with 37.5 μM of sodium acetate, 10 μM of sodium propionate and 2.5 μM of sodium butyrate; SCFA 60 μM=supplemented with 45 μM of sodium acetate, 12 μM of sodium propionate and 3 μM of sodium butyrate.

FIG. 3—Effect of human milk oligosaccharides (HMOs) on vitamin K2 production in a gut model

The following groups were evaluated: Blank; Single HMO (1.3 g/L 2FL); HMO mix (2.5 g/L 2FL, DiFL, LNnT, LNT, and 6SL); B. infantis (10⁷ cfu/ml); Single HMO+B. infantis; HMO mix+B. infantis. (B) The following groups were evaluated (in a milk matrix): w/o LPR (no L. rhamnosus LPR added); and w/LPR (4.5×10⁷ cfu/ml L. rhamnosus LPR).

FIG. 4—Preclinical experimental design

Faltering growth group (with vit. K1): the number of pups per BALB/c mother has been increased by 50% from D8 to D18 to induce a food restriction. In both groups (normal and faltering growth), weaning happened at D18. Male and female mice then received food ad libitum and a daily nutritional supplementation through pipet feeding for 30 days.

FIG. 5A&B—Impact of Vit. K2AD+synbiotic [2′-fucosyllactose+B. infantis] on tibia growth plate cartilage assessed through histological analysis.

Tibias were placed in separate histological cassette between foams and were fixed in 10% (v/v) neutral buffered formalin at room temperature for at least 48 h, decalcified for a minimum of 4 h in DC2 (if necessary), and then were embedded in paraffin until further histological processing. Two sagittal sections (5 μm) were cut with a microtome and stained according to a standard haematoxylin and eosin (H&E) protocol. A histomorphometry analysis of the resting, proliferative, pre-hypertrophic, and hypertrophic zones were performed to evaluate the total growth plate height as well as the height of each of the 4 precited zones (Guevara-Morales J M, Frohbergh M, Castro-Abril H, et al. Growth Plate Pathology in the Mucopolysaccharidosis Type VI Rat Model—An Experimental and Computational Approach. Diagnostics. 2020; 10 (6). doi: 10.3390/diagnostics10060360; Wilson K, Usami Y, Hogarth D, et al. Analysis of Association between Morphometric Parameters of Growth Plate and Bone Growth of Tibia in Mice and Humans. Cartilage. 2020; 13 (2_suppl): 315S-325S. doi: 10.1177/1947603519900800).

FIG. 6—Impact of Vit. K2AD+/−synbiotic [2′-fucosyllactose+B. infantis] on trabecular connectivity density (Conn. D) and trabecular number (Tb.N) assessed through micro-computed tomography on femurs

Micro-computed tomography (μCT UCT35, Scanco Medical AG, Basserdorf, Switzerland) was used to assess trabecular microstructure at distal metaphysis of femurs, as previously described (N. Bonnet, J. Brun, J. C. Rousseau, L. T. Duong, S. L. Ferrari, Cathepsin K Controls Cortical Bone Formation by Degrading Periostin, J. Bone Miner. Res., 2017, 32 (7): 1432-1441). Briefly, trabecular bone region was evaluated using isotropic 6 μm voxels. To eliminate the primary spongiosa, 30 slices of bone under the distal growth plate were not considered. The 80 slices of secondary spongiosa directly below were analyzed. Morphometric variables were computed from binarized images using direct, three-dimensional techniques that do not rely on prior assumptions about the underlying structure (N. Bonnet, N. Laroche, L. Vico, E. Dolleans, D. Courteix, C. L. Benhamou, Assessment of trabecular bone microarchitecture by two different x-ray microcomputed tomographs: a comparative study of the rat distal tibia using Skyscan and Scanco devices, Med. Phys., 2009, 36 (4): 1286-97). The connectivity density (Conn. D) and the trabecular number (Tb.N) were assessed.

FIG. 7—Impact of Vit. K2AD+synbiotic [2′-fucosyllactose+B. infantis] on cortical bone mineral density (Ct.BMD) assessed through micro-computed tomography on femurs.

Micro-computed tomography (μCT UCT35, Scanco Medical AG, Basserdorf, Switzerland) was used to assess cortical microstructure at midshaft diaphysis of femurs, as previously described (N. Bonnet, J. Brun, J. C. Rousseau, L. T. Duong, S. L. Ferrari, Cathepsin K Controls Cortical Bone Formation by Degrading Periostin, J. Bone Miner. Res., 2017, 32 (7): 1432-1441). Briefly, cortical bone region was evaluated using isotropic 6 μm voxels. Femoral cortical structure was assessed using 60 continuous CT slides located at the femur midshaft. Morphometric variables were computed from binarized images using direct, three-dimensional techniques that do not rely on prior assumptions about the underlying structure (N. Bonnet, N. Laroche, L. Vico, E. Dolleans, D. Courteix, C. L. Benhamou, Assessment of trabecular bone microarchitecture by two different x-ray microcomputed tomographs: a comparative study of the rat distal tibia using Skyscan and Scanco devices, Med. Phys., 2009, 36 (4): 1286-97). Cortical bone mineral density (Ct.BMD) was assessed.

FIG. 8—Impact of Vit. K2AD+synbiotic [2′-fucosyllactose+B. infantis] on femur strength assessed through axial compression of the metaphysis.

1.7 mm high slices were cut from the femurs to isolate the metaphysis. The metaphysis slices were compressed axially to test the biomechanical properties of the femur as previously described (C. H. Turner, D. B. Burr, Basic biomechanical measurements of bone: a tutorial, Bone, 1993, 14 (4): 595-608). The load was applied in a compression mode at a nominal deformation rate of 2 mm/min until fracture. Load-displacement curves were recorded to determine the elastic energy.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

Any reference to prior art documents in this specification is not to be considered an admission that such prior art is widely known or forms part of the common general knowledge in the field. All publications mentioned in the specification are herein incorporated by reference.

As used in this specification, the words “comprises”, “comprising”, and similar words, are not to be interpreted in an exclusive or exhaustive sense. In other words, they are intended to mean “including, but not limited to”. The terms “comprises”, “comprising”, and similar words also include the term “consisting of”.

The practice of the present invention will employ, unless otherwise indicated, conventional techniques which are within the capabilities of one of ordinary skill in the art. Such techniques are explained in the literature. Unless defined otherwise, all technical and scientific terms used herein have the same meaning as commonly understood by one of ordinary skill in the art.

Numeric ranges are inclusive of the numbers defining the range and all percentages disclosed herein are on a w/w basis, unless stated otherwise. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

### Combination

In one aspect, the present invention provides a combination of a vitamin mixture and a synbiotic for use in enhancing bone growth and/or bone strength in a subject.

The combination of the present invention may be referred to as a combination therapy. As used herein, a “combination therapy” may refer to a therapy comprising the administration of two or more agents, mixtures (e.g. a vitamin mixture, an oligosaccharide mixture, and/or a probiotic mixture), or compositions (e.g. a vitamin mixture, an oligosaccharide mixture, and/or a probiotic).

The combination may be administered by any suitable route and in any suitable form. Suitably, the combination is administered by oral and/or enteral administration. In preferred embodiments, the combination is administered by oral administration. The combination may be administered separately, simultaneously or sequentially. In preferred embodiments, the combination is administered simultaneously.

### Vitamin Mixture

The combination of the present invention comprises a vitamin mixture. As used herein, a “vitamin mixture” may refer to a mixture of two or more vitamins. Vitamins are organic micronutrients required by the body to carry out a range of normal functions and include vitamin K2, vitamin A, vitamin D, vitamin C, folate, vitamin B3, vitamin B6, vitamin B12, and vitamin E.

The vitamin mixture used in the present invention comprises or consists of vitamin K2, vitamin A, and vitamin D. The present inventors have surprisingly found that a combination of vitamin K2, vitamin A, and vitamin D synergistically promotes osteoblast mineralization.

### Vitamin K2

Vitamin K2 (menaquinone) is one of three types of vitamin K, the other two being vitamin K1 (phylloquinone) and K3 (menadione) and may have the general formula below:

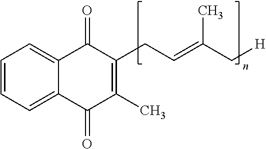

Vitamin K2 consists of various forms, differing in the number (n) of isoprenyl units, wherein n may range from 4 to 13. The various forms are indicated by a suffix (-n), for example menaquinone-7 (MK-7) has seven isoprenyl units (n=7).

Suitably, the vitamin K2 comprises of consists of menaquinone-4 (MK-4), menaquinone-5 (MK-5), menaquinone-6 (MK-6), menaquinone-7 (MK-7), menaquinone-8 (MK-8) and menaquinone-9 (MK-9). Suitably, the vitamin K2 comprises of consists of menaquinone-7 (MK-7), menaquinone-8 (MK-8) and menaquinone-9 (MK-9). In some embodiments, the vitamin K2 comprises of consists of menaquinone-7 (MK-7). In some embodiments, the vitamin K2 consists of menaquinone-7 (MK-7).

The present inventors have surprisingly found that vitamin K2 production in the gastrointestinal tract (e.g. via conversion of vitamin K1) is promoted by a synbiotic.

In one aspect, the present invention provides use of a synbiotic to promote vitamin K2 production in the gut of a subject.

In one aspect, the present invention provides a method for promoting vitamin K2 production in the gut of a subject, the method comprising administering to the subject an effective amount of a synbiotic.

The synbiotic may be any described herein. In some embodiments, the synbiotic comprises one or more oligosaccharide and one or more probiotic. For example, the synbiotic may comprise 2′-fucosyllactose (2′FL) and one or more probiotic. For example, the synbiotic may comprise 2′-fucosyllactose (2′FL) and Bifidobacterium infantis.

In some embodiments, the synbiotic promotes de novo menaquinone-7 production in the subject's gut (e.g. by the gut microbiota). In some embodiments, the synbiotic promotes bioconversion from phylloquinone to menaquinone-4 in the subject's gut (e.g. by the gut microbiota).

### Vitamin A

Vitamin A comprises a family of molecules containing a 20 carbon structure with a methyl substituted cyclohexenyl ring (beta-ionone ring) and a tetraene side chain with a hydroxyl group (retinol), aldehyde group (retinal), carboxylic acid group (retinoic acid), or ester group (retinyl ester) at carbon-15. The term vitamin A may also include provitamin A carotenoids that are dietary precursors of retinol. Of the many carotenoids in nature, several have provitamin A nutritional activity, including α-carotene, β-carotene, and β-cryptoxanthin.

The amount of vitamin A may be referred to in retinol equivalents (RE) or retinol activity equivalents (RAE). For dietary provitamin A carotenoids β-carotene, α-carotene, and β-cryptoxanthin, REs have been set at 6, 12, and 12 μg, respectively. Using μg RAE, the vitamin A activity of provitamin A carotenoids is half the vitamin A activity assumed when using μg RE. For dietary provitamin A carotenoids β-carotene, α-carotene, and β-cryptoxanthin, RAEs have been set at 12, 24, and 24 μg, respectively. (See e.g. Dietary Reference Intakes for Vitamin A, Vitamin K, Arsenic, Boron, Chromium, Copper, Iodine, Iron, Manganese, Molybdenum, Nickel, Silicon, Vanadium, and Zinc. Washington (DC): National Academies Press (US); 2001. 4, Vitamin A).

### Vitamin D

Vitamin D is a group of fat-soluble secosteroids including vitamin D2 (ergocalciferol) and vitamin D3 (cholecalciferol). Calcitriol (also known as 1,25-dihydroxyvitamin D) is the active form of vitamin D. Suitably, the vitamin D comprises or consists of vitamin D2 and vitamin D3. Suitably, the vitamin D comprises or consists of calcitriol.

### Administration of Vitamins K2, a, and D

The subject may be administered any suitable amounts of vitamins K2, A, and D, in any suitable form and via any suitable route of administration (e.g. in any form and via any route described herein).

Suitable doses of vitamin K2 are described in e.g. Kozioł-Kozakowska, A. and Maresz, K., 2022. Children, 9 (1), p. 78 and European Food Safety Authority (EFSA), 2008. EFSA Journal, 6 (11), p. 822. Suitable doses of vitamin A are described in e.g. Ross, A. C. and Moran, N. E., 2020. Current Developments in Nutrition, 4 (10), p.nzaa096 and EFSA Panel on Dietetic Products, Nutrition, and Allergies (NDA), 2015. EFSA Journal, 13 (3), p. 4028. Suitable doses of vitamin D are described in e.g. Greer, F. R., 2004. The American journal of clinical nutrition, 80 (6), pp. 1759S-1762S and EFSA Panel on Dietetic Products, Nutrition and Allergies (NDA), 2016. EFSA Journal, 14 (10), p. e04547.

Suitably, vitamin K2 is administered to the subject in an amount of at least about 0.5 μg/kg/day, at least about 1 μg/kg/day, or at least about 2 μg/kg/day. Suitably, vitamin K2 is administered to the subject in an amount of about 10 μg/kg/day or less, about 7.5 μg/kg/day or less, or about 5 μg/kg/day or less. Suitably, vitamin K2 is administered to the subject in an amount of from about 0.5 μg/kg/day to about 10 μg/kg/day, from about 1 μg/kg/day to about 7.5 μg/kg/day or from about 2 μg/kg/day to about 5 μg/kg/day.

Suitably, vitamin K2 is administered to the subject in an amount of at least about 5 μg/day, at least about 10 μg/day, at least about 15 μg/day, at least about 20 μg/day, at least about 25 μg/day, or at least about 30 μg/day. Suitably, vitamin K2 is administered to the subject in an amount of about 200 μg/day or less, about 100 μg/day or less, about 90 μg/day or less, about 80 μg/day or less, about 70 μg/day or less, or about 60 μg/day or less. Suitably, vitamin K2 is administered to the subject in an amount of from about 5 μg/day to about 200 μg/day, from about 10 μg/day to about 100 μg/day, from about 15 μg/day to about 90 μg/day, from about 20 μg/day to about 80 μg/day, from about 25 μg/day to about 70 μg/day, or from about 30 μg/day to about 60 μg/day.

Suitably, vitamin A is administered to the subject in an amount of at least about 100 μg RE/day, at least about 200 μg RE/day, or at least about 300 μg RE/day. Suitably, vitamin A is administered to the subject in an amount of about 1000 μg RE/day or less, about 800 μg RE/day or less, about 600 μg RE/day or less, or about 400 μg RE/day or less. Suitably, vitamin A is administered to the subject in an amount of from about 100 μg RE/day to about 1000 μg RE/day, from about 200 μg RE/day to about 800 μg RE/day, from about 300 μg RE/day to about 600 μg RE/day, or from about 300 μg RE/day to about 400 μg RE/day.

Suitably, vitamin A is administered to the subject in an amount of at least about 100 μg RE/day, at least about 200 μg RAE/day, or at least about 300 μg RAE/day. Suitably, vitamin A is administered to the subject in an amount of about 1000 μg RAE/day or less, about 800 μg RAE/day or less, about 600 μg RAE/day or less, or about 400 μg RAE/day or less. Suitably, vitamin A is administered to the subject in an amount of from about 100 μg RAE/day to about 1000 μg RAE/day, from about 200 μg RAE/day to about 800 μg RAE/day, from about 300 μg RAE/day to about 600 μg RAE/day, or from about 300 μg RAE/day to about 400 μg RAE/day.

Suitably, vitamin D is administered to the subject in an amount of at least about 2.5 μg/day, at least about 5 μg/day, at least about 10 μg/day, or at least about 15 μg/day. Suitably, vitamin D is administered to the subject in an amount of about 100 μg/day or less, about 75 μg/day or less, or about 50 μg/day or less. Suitably, vitamin D is administered to the subject in an amount of from about 2.5 μg/day to about 100 μg/day, from about 5 μg/day to about 100 μg/day, from about 10 μg/day to about 75 μg/day or from about 15 μg/day to about 50 μg/day. Suitably, vitamin D is administered to the subject in an amount of about 15 μg/day.

Suitably, vitamin K2 is administered to the subject in an amount of from about 5 μg/day to about 200 μg/day, vitamin A is administered to the subject in an amount of from about 100 μg RE/day to about 1000 μg RE/day, and vitamin D is administered to the subject in an amount of from about 2.5 μg/day to about 100 μg/day.

### Synbiotic

The combination of the present invention comprises a synbiotic. As used herein, the term “synbiotic” may refer to a component that contains both probiotics and prebiotics (see e.g. Swanson, K. S., et al., 2020. Nature Reviews Gastroenterology & Hepatology, 17 (11), pp. 687-701).

In preferred embodiments, the synbiotic comprises or consists of 2′-fucosyllactose (2′FL) and one or more probiotic. In other preferred embodiments, the synbiotic comprises or consists of an oligosaccharide mixture and one or more probiotic.

### Prebiotics

The synbiotic used in the combination of the present invention may comprise one or more prebiotic. As used herein, the term “prebiotic” may refer to a non-digestible component that benefits the subject by selectively stimulating the favourable growth and/or activity of one or more microbial taxa. Exemplary prebiotics include human milk oligosaccharides. Exemplary prebiotic oligosaccharides include galacto-oligosaccharides (GOS), fructo-oligosaccharides (FOS), 2′-fucosyllactose, lacto-N-neo-tetraose, and inulin.

In some embodiments, the synbiotic comprises one or more oligosaccharide. Oligosaccharides are saccharide polymers containing a small number (typically two to ten) of monosaccharides and are one of the best know prebiotics. Short chain fatty acids (SCFAs) such as acetate, butyrate, and propionate may be produced by fermentation of oligosaccharides in the gut. In some embodiments, the synbiotic comprises an oligosaccharide mixture. As used herein, an “oligosaccharide mixture” may refer to a mixture of two or more oligosaccharides.

The one or more oligosaccharide used in the present invention may comprise or consist of human milk oligosaccharides (HMOs). Many different kinds of HMOs are found in the human milk and are typically based on a combination of glucose, galactose, sialic acid (N-acetylneuraminic acid), fucose and/or N-acetylglucosamine with many and varied linkages between them. Almost all HMOs have a lactose moiety at their reducing end while sialic acid and/or fucose (when present) occupy terminal positions at the non-reducing ends. HMOs can be acidic (e.g. charged sialic acid containing oligosaccharides) or neutral (e.g. fucosylated oligosaccharides).

Suitably, the one or more oligosaccharide may comprise at least one fucosylated oligosaccharide, at least one sialylated oligosaccharide, and/or at least one N-acetylated oligosaccharide. In some embodiments, the one or more oligosaccharide comprises or consists of at least one fucosylated oligosaccharide, at least one sialylated oligosaccharide, and at least one N-acetylated oligosaccharide. In some embodiments, the one or more oligosaccharide comprises or consists of 2′-fucosyllactose (2′FL), difucosyllactose (diFL), 6′-sialyllactose (6′-SL), lacto-N-tetraose (LNT), and lacto-N-neotetraose (LNnT). In some embodiments, the one or more oligosaccharide comprises or consists of 2′-fucosyllactose (2′FL), difucosyllactose (diFL), 6′-sialyllactose (6′-SL), 3′-sialyllactose (3′-SL), lacto-N-tetraose (LNT), and lacto-N-neotetraose (LNnT).

In some embodiments, the one or more oligosaccharide comprises about 30 to about 80 wt %, about 40 to about 80 wt %, or about 50 to about 70 wt %, with respect to the total weight of the oligosaccharides, of at least one fucosylated oligosaccharide. In some embodiments, the one or more oligosaccharide comprises about 10 to about 35 wt %, about 10 to about 30 wt %, or about 10 to about 25 wt %, with respect to the total weight of the oligosaccharides, of at least one sialylated oligosaccharide. In some embodiments, the one or more oligosaccharide comprises about 10 to about 35 wt %, about 15 to about 30 wt %, or about 15 to about 20 wt %, with respect to the total weight of the oligosaccharides, of at least one N-acetylated oligosaccharide.

In some embodiments, the one or more oligosaccharide comprises or consists of about 30 to about 80 wt %, about 40 to about 80 wt %, or about 50 to about 70 wt %, with respect to the total weight of the oligosaccharides, of at least one fucosylated oligosaccharide; and about 10 to about 35 wt %, about 15 to about 30 wt %, or about 15 to about 20 wt %, with respect to the total weight of the oligosaccharides, of at least one N-acetylated oligosaccharide.

In some embodiments, the one or more oligosaccharide comprises or consists of: about 30 to about 80 wt %, about 40 to about 80 wt %, or about 50 to about 70 wt %, with respect to the total weight of the oligosaccharides, of at least one fucosylated oligosaccharide; about 10 to about 35 wt %, about 10 to about 30 wt %, or about 10 to about 25 wt %, with respect to the total weight of the oligosaccharides, of at least one sialylated oligosaccharide; and about 10 to about 35 wt %, about 15 to about 30 wt %, or about 15 to about 20 wt %, with respect to the total weight of the oligosaccharides, of at least one N-acetylated oligosaccharide.

The oligosaccharides may be obtained by any suitable method. Suitable methods for synthesising oligosaccharides will be well known to those of skill in the art. For example, processes have been developed for producing oligosaccharides by microbial fermentations, enzymatic processes, chemical syntheses, or combinations of these technologies (see e.g. Zeuner et al., 2019. Molecules, 24 (11), p. 2033).

### Fucosylated Oligosaccharides

In some embodiments, the one or more oligosaccharide comprises or consists of at least one fucosylated oligosaccharide.

Non-limiting example(s) of fucosylated oligosaccharide(s) include: 2′-fucosyllactose (2′FL), 3-fucosyllactose (3FL), difucosyllactose (diFL), lacto-N-fucopentaose, such as lacto-N-fucopentaose I (LNFP-I), lacto-N-fucopentaose II (LNFP-II), lacto-N-fucopentaose III (LNFP-III) or lacto-N-fucopentaose V (LNFP-V), lacto-N-fucohexaose, lacto-N-difucohexaose I, lacto-neofucopentaose V (LNnFP-V), lacto-N-difucosylhexaose-I (LNDFH-1), lacto-N-neodifucosylhexaose (LNnDFH), fucosyllacto-N-hexaose, fucosyllacto-N-neohexaose (such as fucosyllacto-N-neohexaose I, fucosyllacto-N-neohexaose II), monofucosyllacto-n-hexaose-III (MFNLH-III), difucosyllacto-N-hexaose I, difuco-lacto-N-neohexaose, difucosyllacto-N-neohexaose I, difucosyllacto-N-neohexaose II, difucosyllacto-N-hexaose-a (DFLNHa), fucosyl-para-Lacto-N-hexaose, tri-fuco-para-Lacto-N-hexaose I, and combinations thereof.

In preferred embodiments, the at least one fucosylated oligosaccharide comprises or consists of 2′-fucosyllactose (2′FL), which is typically the most prevalent HMO naturally present in human breast milk.

In some embodiments, the at least one fucosylated oligosaccharide is selected from the group consisting of 2′-fucosyllactose (2′FL), difucosyllactose (diFL) and combinations thereof. In some embodiments, the at least one fucosylated oligosaccharide comprises or consists of 2′-fucosyllactose (2′FL) and difucosyllactose (diFL).

The fucosylated oligosaccharides may be obtained by any suitable method. For example, 2′FL may be produced by biotechnological means using specific fucosyltransferases and/or fucosidases either through the use of enzyme-based fermentation technology (recombinant or natural enzymes) or microbial fermentation technology. In the latter case, microbes may either express their natural enzymes and substrates or may be engineered to produce respective substrates and enzymes. Alternatively, 2′FL may be produced by chemical synthesis from lactose and free fucose. diFL may be synthesized by enzymatic, biotechnological and/or chemical processes.

### Sialylated Oligosaccharides

In some embodiments, the one or more oligosaccharide comprises at least one sialylated oligosaccharide.

Non-limiting examples of sialylated oligosaccharides include: 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof.

In some embodiments, the at least one sialylated oligosaccharide is selected from the group consisting of 3′-sialyllactose (3′-SL), 6′-sialyllactose (6′-SL) and combinations thereof. In some embodiments, the at least one sialylated oligosaccharide comprises or consists of 6′-sialyllactose (6′-SL). In some embodiments, the at least one sialylated oligosaccharide comprises or consists of 6′-sialyllactose (6′-SL) and 3′-sialyllactose (3′-SL).

The sialylated oligosaccharides may be obtained by any suitable method. For example, 3′-sialyllactose (3′-SL), and/or 6′-sialyllactose (6′-SL) may be isolated by chromatographic or filtration technology from a natural source such as animal milks. Alternatively, they may be produced by biotechnological means using specific sialyltransferases or sialidases, neuraminidases, either by an enzyme based fermentation technology (recombinant or natural enzymes), by chemical synthesis or by a microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerisation (DP), from DP=1 onwards. Alternatively, sialyllactoses may be produced by chemical synthesis from lactose and free N′-acetylneuraminic acid (sialic acid). Sialyllactoses are also commercially available for example from Kyowa Hakko Kogyo, Japan, or from GeneChem, Republic of Korea.

If the one or more oligosaccharide comprises 3′-Sialyllactose (3′-SL) and 6′-Sialyllactose (6′-SL), it may be particularly beneficial if said 3′-Sialyllactose (3′-SL) and 6′-Sialyllactose (6′-SL) are comprised in said nutritional composition in a weight ratio between about 10:1 and about 1:10, such as between about 10:1 and about 2:1, between about 8:1 and about 3:1, between about 6:1 and about 3:1, between about 5:1 and about 3:1, between about 5:1 and about 4:1, or between about 1:2 and about 1.5:1.

### N-Acetylated Oligosaccharides

In some embodiments, the one or more oligosaccharide comprises at least one N-acetylated oligosaccharide.

Suitably, the at least one N-acetylated oligosaccharide is selected from the group consisting of N-acetyl-glucosamine, N-acetyl-galactosamines and combinations thereof. Non-limiting examples of N-acetylated oligosaccharide(s) include: LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

In some embodiments, the at least one N-acetylated oligosaccharide is selected from the group consisting of lacto-N-tetraose (LNT), lacto-N-neotetraose (LNnT) and combinations thereof. In some embodiments, the at least one N-acetylated oligosaccharide comprises or consists of lacto-N-tetraose (LNT) and lacto-N-neotetraose (LNnT).

The N-acetylated oligosaccharides may be obtained by any suitable method. For example, LNnT may be synthesised chemically by enzymatic transfer of saccharide units from donor moieties to acceptor moieties using glycosyltransferases. Alternatively, LNnT may be prepared by chemical conversion of Keto-hexoses (e.g. fructose) either free or bound to an oligosaccharide (e.g. lactulose) into N-acetylhexosamine or an N-acetylhexosamine-containing oligosaccharide. LNT may be synthesized by enzymatic, biotechnological and/or chemical processes.

### Administration of Oligosaccharides

The subject may be administered any suitable amounts of oligosaccharides, in any suitable form and via any suitable route of administration (e.g. in any form and via any route described herein).

Suitable doses of human oligosaccharides are described in e.g. EFSA Panel on Dietetic Products, Nutrition and Allergies (NDA), 2015. EFSA Journal, 13 (11), p. 4299; EFSA Panel on Nutrition, Novel Foods and Food Allergens (NDA), 2019. EFSA Journal, 17 (6), p.e05717; EFSA Panel on Nutrition, Novel Foods and Food Allergens (NDA), 2020. EFSA Journal, 18 (5), p.e06097; EFSA Panel on Nutrition, Novel Foods and Food Allergens (NDA), 2022. EFSA Journal, 20 (5), p.e07331; and EFSA Panel on Nutrition, Novel Foods and Food Allergens (NDA), 2019. EFSA Journal, 17 (12), p.e05907.

Suitably, the one or more oligosaccharide is administered to the subject in an amount of at least about 0.5 g/day, at least about 1 g/day, or at least about 2 g/day. Suitably, the one or more oligosaccharide is administered to the subject in an amount of about 10 g/day or less, about 8 g/day or less, or about 5 g/day or less. Suitably, the one or more oligosaccharide is administered to the subject in an amount of from about 0.5 g/day to about 10 g/day, from about 1 g/day to about 8 g/day or from about 2 g/day to about 5 g/day.

Suitably, 2′FL is administered to the subject in an amount of at least about 0.5 g/day, at least about 0.8 g/day, or at least about 1 g/day. Suitably, 2′FL is administered to the subject in an amount of about 5 g/day or less, about 4 g/day or less, or about 3 g/day or less. Suitably, 2′FL is administered to the subject in an amount of from about 0.5 g/day to about 5 g/day, from about 0.8 g/day to about 4 g/day or from about 1 g/day to about 3 g/day.

Suitably, diFL is administered to the subject in an amount of at least about 0.1 g/day, at least about 0.15 g/day, or at least about 0.2 g/day. Suitably, diFL is administered to the subject in an amount of about 1 g/day or less, about 0.8 g/day or less, or about 0.6 g/day or less. Suitably, diFL is administered to the subject in an amount of from about 0.1 g/day to about 1 g/day, from about 0.15 g/day to about 0.8 g/day or from about 0.2 g/day to about 0.6 g/day.

Suitably, 6′-SL is administered to the subject in an amount of at least about 0.1 g/day, at least about 0.15 g/day, or at least about 0.2 g/day. Suitably, 6′-SL is administered to the subject in an amount of about 1 g/day or less, about 0.8 g/day or less, or about 0.6 g/day or less. Suitably, 6′-SL is administered to the subject in an amount of from about 0.1 g/day to about 1 g/day, from about 0.15 g/day to about 0.8 g/day or from about 0.2 g/day to about 0.6 g/day.

Suitably, 3′-SL is administered to the subject in an amount of at least about 0.1 g/day, at least about 0.15 g/day, or at least about 0.2 g/day. Suitably, 3′-SL is administered to the subject in an amount of about 1 g/day or less, about 0.8 g/day or less, or about 0.6 g/day or less. Suitably, 3′-SL is administered to the subject in an amount of from about 0.1 g/day to about 1 g/day, from about 0.15 g/day to about 0.8 g/day or from about 0.2 g/day to about 0.6 g/day.

Suitably, LNT is administered to the subject in an amount of at least about 0.2 g/day, at least about 0.3 g/day, or at least about 0.4 g/day. Suitably, LNT is administered to the subject in an amount of about 2.5 g/day or less, about 2 g/day or less, or about 1.5 g/day or less. Suitably, LNT is administered to the subject in an amount of from about 0.2 g/day to about 2.5 g/day, from about 0.3 g/day to about 2 g/day or from about 0.4 g/day to about 1.5 g/day.

Suitably, LNnT is administered to the subject in an amount of at least about 0.2 g/day, at least about 0.3 g/day, or at least about 0.4 g/day. Suitably, LNnT is administered to the subject in an amount of about 2.5 g/day or less, about 2 g/day or less, or about 1.5 g/day or less. Suitably, LNnT is administered to the subject in an amount of from about 0.2 g/day to about 2.5 g/day, from about 0.3 g/day to about 2 g/day or from about 0.4 g/day to about 1.5 g/day.

Suitably, vitamin K2 is administered to the subject in an amount of from about 5 μg/day to about 200 μg/day, vitamin A is administered to the subject in an amount of from about 100 μg RE/day to about 1000 μg RE/day, vitamin D is administered to the subject in an amount of from about 2.5 μg/day to about 100 μg/day, and the one or more oligosaccharide is administered to the subject in a total amount of from about 0.5 g/day to about 10 g/day. Suitably, 2′FL is administered to the subject in an amount of from about 0.5 g/day to about 5 g/day; diFL is administered to the subject in an amount of from about 0.1 g/day to about 1 g/day; 6′-SL is administered to the subject in an amount of from about 0.1 g/day to about 1 g/day; 3′-SL is administered to the subject in an amount of from about 0.1 g/day to about 1 g/day; LNT is administered to the subject in an amount of from about 0.2 g/day to about 2.5 g/day; and/or LNnT is administered to the subject in an amount of from about 0.2 g/day to about 2.5 g/day.

Suitably, vitamin K2 is administered to the subject in an amount of from about 5 μg/day to about 200 μg/day, vitamin A is administered to the subject in an amount of from about 100 μg RE/day to about 1000 μg RE/day, vitamin D is administered to the subject in an amount of from about 2.5 μg/day to about 100 μg/day, and 2′FL is administered to the subject in an amount of from about 0.5 g/day to about 5 g/day.

### Probiotics

The synbiotic used in the combination of the present invention comprises one or more probiotic. As used herein, the term “probiotic” may refer to a component that contains a sufficient number of viable microorganisms to alter the gut microbiota of the subject (see e.g. Hill, C., et al., 2014. Nature reviews Gastroenterology & hepatology, 11 (8), p. 506). Suitably, the one or more probiotic comprises one or more commercially available probiotic strain and/or a strain which has been shown to have health benefits (See e.g. Fijan, S., 2014. International journal of environmental research and public health, 11 (5), pp. 4745-4767). Exemplary probiotic microorganisms may include Bifidobacterium, Lactobacillus, Limosilactobacillus, Lacticaseibacillus, Saccharomyces, Enterococcus, Streptococcus, Pediococcus, Leuconostoc, Bacillus, and Escherichia coli. In some embodiments, the synbiotic comprises a probiotic mixture. As used herein, an “probiotic mixture” may refer to a mixture of two or more probiotics.

In some embodiments, the one or more probiotic comprises or consists of Bifidobacterium, Lactobacillus, Limosilactobacillus, and/or Lacticaseibacillus.

In some embodiments, the one or more probiotic comprises or consists of Bifidobacterium longum, Bifidobacterium animalis and/or Lactobacillus rhamnosus. In some embodiments, the one or more probiotic comprises or consists of Bifidobacterium longum and/or Bifidobacterium animalis. In some embodiments, the one or more probiotic comprises or consists of Bifidobacterium longum and/or Bifidobacterium animalis.

Bifidobacterium longum

In preferred embodiments, the one or more probiotic comprises or consists of Bifidobacterium longum.

Bifidobacterium longum is a bacterium present in the human gastrointestinal tract. In 2002, three previously distinct species of Bifidobacterium, B. infantis, B. longum, and B. suis, were unified into a single species named B. longum with the biotypes infantis, longum, and suis, respectively (Sakata, S., et al., 2002. International journal of systematic and evolutionary microbiology, 52 (6), pp. 1945-1951).

In some embodiments, the one or more probiotic comprises or consists of Bifidobacterium longum ssp. infantis (also known as Bifidobacterium infantis), Bifidobacterium longum ssp. suis (also known as Bifidobacterium suis), and/or Bifidobacterium longum ssp. longum (also known as Bifidobacterium longum).

In preferred embodiments, the one or more probiotic comprises or consists of Bifidobacterium infantis. In some embodiments, the one or more probiotic comprises or consists of Bifidobacterium infantis LMG 11588 or a derivative thereof (e.g. R0033 which has been communicated to the US Food and Drug Administration as generally recognized as safe (GRAS), see e.g. Duboux, S., et al., 2022. Microorganisms, 10 (2), p. 203). B. infantis may boost endogenous production of vitamin K2, through the production of precursors.

Bifidobacterium animalis

In some embodiments, the one or more probiotic comprises or consists of Bifidobacterium animalis.

Bifidobacterium animalis is a bacterium of the Bifidobacterium genus which can be found in the large intestines of most mammals, including humans. Bifidobacterium animalis and Bifidobacterium lactis were previously described as two distinct species. Presently, both are considered B. animalis with the subspecies Bifidobacterium animalis subsp. animalis and Bifidobacterium animalis subsp. lactis (see Masco, L., et al., 2004. International Journal of Systematic and Evolutionary Microbiology, 54 (4), pp. 1137-1143).

In some embodiments, the one or more probiotic comprises or consists of Bifidobacterium animalis ssp. lactis (also known as Bifidobacterium lactis). For example, the bacterium, Bifidobacterium lactis HN019, has been studied for a variety of traits important to its ability to function as a probiotic (see e.g. Sanders, M. E., 2006. Journal of clinical gastroenterology, 40 (9), pp. 776-783).

Lactobacillus rhamnosus

In some embodiments, the one or more probiotic comprises or consists of Lactobacillus rhamnosus.

Lacticaseibacillus rhamnosus (also known as Lactobacillus rhamnosus) is a short Gram-positive homofermentative facultative anaerobic non-spore-forming rod that often appears in chains. Lactobacillus rhamnosus GG (LGG) is one of the most widely used probiotic strains. Various health effects are well documented (see e.g. Segers, M. E. and Lebeer, S., 2014. Microbial cell factories, 13 (1), pp. 1-16). L. rhamnosus may boost endogenous production of vitamin K2, through the production of precursors.

In some embodiments, the one or more probiotic comprises or consists of Lactobacillus rhamnosus LPR.

### Administration of Probiotics

The subject may be administered any suitable amounts of probiotics, in any suitable form and via any suitable route of administration (e.g. in any form and via any route described herein).

Suitably, the one or more probiotic is administered to the subject in a total amount of at least about 10⁵ cfu/day, at least about 10⁶ cfu/day, at least about 10⁷ cfu/day, at least about 10⁸ cfu/day, at least about 10⁹ cfu/day, or at least about 10¹⁰ cfu/day. Suitably, the one or more probiotic is administered to the subject in a total amount of about 10¹² cfu/day or less, about 10¹¹ cfu/day or less, or about 10¹⁰ cfu/day or less. Suitably, the one or more probiotic is administered to the subject in a total amount of from about 10⁶ cfu/day to about 10¹² cfu/day, from about 10⁷ cfu/day to about 10¹¹ cfu/day or from about 10⁸ cfu/day to about 10¹⁰ cfu/day.

Suitably, Bifidobacterium longum (e.g. B. infantis) is administered to the subject in an amount of at least about 10⁵ cfu/day, at least about 10⁶ cfu/day, at least about 10⁷ cfu/day, at least about 10⁸ cfu/day, at least about 10⁹ cfu/day, or at least about 10¹⁰ cfu/day. Suitably, Bifidobacterium longum (e.g. B. infantis) is administered to the subject in an amount of about 10¹² cfu/day or less, about 10¹¹ cfu/day or less, or about 10¹⁰ cfu/day or less. Suitably, Bifidobacterium longum (e.g. B. infantis) is administered to the subject in an amount of from about 10⁶ cfu/day to about 10¹² cfu/day, from about 10⁷ cfu/day to about 10¹¹ cfu/day or from about 10⁸ cfu/day to about 10¹⁰ cfu/day.

Suitably, Bifidobacterium animalis (e.g. B. lactis) is administered to the subject in an amount of at least about 10⁵ cfu/day, at least about 10⁶ cfu/day, at least about 10⁷ cfu/day, at least about 10⁸ cfu/day, at least about 10⁹ cfu/day, or at least about 10¹⁰ cfu/day. Suitably, Bifidobacterium animalis (e.g. B. lactis) is administered to the subject in an amount of about 10¹² cfu/day or less, about 10¹¹ cfu/day or less, or about 10¹⁰ cfu/day or less. Suitably, Bifidobacterium animalis (e.g. B. lactis) is administered to the subject in an amount of from about 10⁶ cfu/day to about 10¹² cfu/day, from about 10⁷ cfu/day to about 10¹¹ cfu/day or from about 10⁸ cfu/day to about 10¹⁰ cfu/day.

Suitably, Lactobacillus rhamnosus is administered to the subject in an amount of at least about 10⁵ cfu/day, at least about 10⁶ cfu/day, at least about 10⁷ cfu/day, at least about 10⁸ cfu/day, at least about 10⁹ cfu/day, or at least about 10¹⁰ cfu/day. Suitably, Lactobacillus rhamnosus is administered to the subject in an amount of about 10¹² cfu/day or less, about 10¹¹ cfu/day or less, or about 10¹⁰ cfu/day or less. Suitably, Lactobacillus rhamnosus is administered to the subject in an amount of from about 10⁶ cfu/day to about 10¹² cfu/day, from about 10⁷ cfu/day to about 10¹¹ cfu/day or from about 10⁸ cfu/day to about 10¹⁰ cfu/day.

Suitably, vitamin K2 is administered to the subject in an amount of from about 5 μg/day to about 200 μg/day, vitamin A is administered to the subject in an amount of from about 100 μg RE/day to about 10⁰⁰ μg RE/day, vitamin D is administered to the subject in an amount of from about 2.5 μg/day to about 100 μg/day, one or more oligosaccharide is administered to the subject in a total amount of from about 0.5 g/day to about 10 g/day, and one or more probiotic is administered to the subject in a total amount of from about 10⁶ cfu/day to about 10¹² cfu/day.

Suitably, vitamin K2 is administered to the subject in an amount of from about 5 μg/day to about 200 μg/day, vitamin A is administered to the subject in an amount of from about 100 μg RE/day to about 10⁰⁰ μg RE/day, vitamin D is administered to the subject in an amount of from about 2.5 μg/day to about 100 μg/day, 2′FL is administered to the subject in an amount of from about 0.5 g/day to about 5 g/day, and B. infantis is administered to the subject in a total amount of from about 10⁶ cfu/day to about 10¹² cfu/day.

### Compositions

Suitably, the combination is in the form of a composition. The composition may comprise the combination in any therapeutically effective amount.

The composition can be any type of composition in which the combination can be incorporated, such as a composition in the form of a food or beverage product, an animal feed product, a nutritional supplement for human or animal, or a pharmaceutical composition. The composition may be in solid (e.g. powder), liquid or semi-liquid form. The combination may be in the form of a food composition, a pet food composition, a beverage, a nutritional formula, a nutritional supplement, or a nutraceutical.

Food and beverage products include all products intended to be consumed orally by human beings, for the purpose of providing nutrition and/or pleasure. It can for example be a nutritional composition, such as for children. Examples of food and beverage products include dairy products such as milk products or yogurts, soups, sauces, sweet and savoury snacks, powdered drinks and cereal products.

In some embodiments, the combination is in the form of a nutritional composition, a medical food product for clinical nutrition, a milk formula, or a supplement.

In some embodiments, the combination is in the form of a nutritional composition. As used herein, a “nutritional composition” may mean a composition which nourishes a subject. This nutritional composition is usually to be taken orally or intravenously, and typically includes a lipid or fat source and a protein source.

In some embodiments, the combination is in the form of a medical food product for clinical nutrition. As used herein, a “medical food product for clinical nutrition” may also be known as a “Food for Special Medical Purposes (FSMP)” and refer to specialised foods designed to help meet the nutritional or dietary needs of subjects living with a disease, disorder or medical condition who are temporarily or permanently unable to achieve an adequate nutritional intake from normal foods or through modification of the normal diet.

In preferred embodiments, the combination is in the form of a milk formula. The term “milk formula” as used herein may refer to a foodstuff intended for e.g. childhood nutrition, which may provide the sole source or supplemental nutrition for children e.g. aged about 3 years or older. In some embodiments, the milk formula is a grow milk.

In some embodiments, the composition (e.g. milk formula) is in a powder form and reconstituted in an aqueous medium (e.g. water) prior to administration. In other embodiments, the composition (e.g. milk formula) is in a liquid form ready for administration (e.g. a ready-to-feed formula).

In another embodiment, the combination is in the form of a supplement. As used herein, a “supplement” or “dietary supplement” may be used to complement the nutrition of a subject (it is typically used as such but it might also be added to any kind of compositions intended to be ingested by the subject). When the composition is a supplement, it can be provided in the form of unit doses. Supplements are typically present in the form of a liquid, a gel, a powder or a tablet or capsule. Powder supplements typically encompass supplements to be dissolved in water or milk, or to be sprinkled on food or in a beverage. Such supplements are intended to provide additional nutrients and/or a health benefit to the subject consuming it. A supplement can be used for providing nutrients and/or a health benefit to human beings, as well as to animals.

In another embodiment, the combination is in the form of a fortifier. The fortifier can be a milk formula fortifier.

In another embodiment, the combination is in the form of a pharmaceutical product. Pharmaceutical products include for example drops, syrups, powder, tablet or capsule products intended to treat of prevent an adverse medical condition in a subject in need thereof.

The combination can also be in the form of an animal food product or a nutritional supplement for animals. Preferably, the animal is a mammal. Examples of animals include primates (e.g., humans), cows, sheep, goats, horses, dogs, cats, rabbits, rats, mice, fish, birds and the like.

A nutritional composition of the invention, and especially the milk formula, generally contains a protein source, a carbohydrate source and a lipid source. In some embodiments however, especially if a nutritional composition of the invention is a supplement or a fortifier, there may be only lipids (or a lipid source).

The nutritional composition of the invention may comprise about 100 kcal/100 g to about 1000 kcal/100 g, about 200 kcal/100 g to about 800 kcal/100 g, or about 400 kcal/100 g to about 600 kcal/100 g, on a dry weight basis.

### Vitamin Mixture

A nutritional composition (e.g. milk formula) according to the invention may contain any suitable amounts of vitamins K2, A, and D.

Suitably, the nutritional composition comprises vitamin K2 in an amount of at least about 5 μg/100 g, at least about 10 μg/100 g, at least about 15 μg/100 g, at least about 20 μg/100 g, at least about 25 μg/100 g, or at least about 30 μg/100 g on a dry weight basis. Suitably, the nutritional composition comprises vitamin K2 in an amount of about 2100 μg/100 g or less, about 100 μg/100 g or less, about 90 μg/100 g or less, about 80 μg/100 g or less, about 70 μg/100 g or less, or about 60 μg/100 g or less, on a dry weight basis. Suitably, the nutritional composition comprises vitamin K2 in an amount of from about 5 μg/100 g to about 200 μg/100 g, from about 10 μg/100 g to about 100 μg/100 g, from about 15 μg/100 g to about 90 μg/100 g, from about 20 μg/100 g to about 80 μg/100 g, from about 25 μg/100 g to about 70 μg/100 g, or from about 30 μg/100 g to about 60 μg/100 g.

Suitably, the nutritional composition comprises vitamin A in an amount of at least about 100 μg RE/100 g, at least about 200 μg RE/100 g, or at least about 300 μg RE/100 g, on a dry weight basis. Suitably, the nutritional composition comprises vitamin A in an amount of about 1000 μg RE/100 g or less, about 800 μg RE/100 g or less, about 600 μg RE/100 g or less, or about 400 μg RE/100 g or less, on a dry weight basis. Suitably, the nutritional composition comprises vitamin A in an amount of from about 100 μg RE/100 g to about 1000 μg RE/100 g, from about 200 μg RE/100 g to about 800 μg RE/100 g, from about 300 μg RE/100 g to about 600 μg RE/100 g, or from about 300 μg RE/100 g to about 400 μg RE/100 g, on a dry weight basis.

Suitably, the nutritional composition comprises vitamin A in an amount of at least about 100 μg RAE/100 g, at least about 200 μg RAE/100 g, or at least about 300 μg RAE/100 g, on a dry weight basis. Suitably, the nutritional composition comprises vitamin A in an amount of about 1000 μg RAE/100 g or less, about 800 μg RAE/100 g or less, about 600 μg RAE/100 g or less, or about 400 μg RAE/100 g or less, on a dry weight basis. Suitably, the nutritional composition comprises vitamin A in an amount of from about 100 μg RAE/100 g to about 1000 μg RAE/100 g, from about 200 μg RAE/100 g to about 800 μg RAE/100 g, from about 300 μg RAE/100 g to about 600 μg RAE/100 g, or from about 300 μg RAE/100 g to about 400 μg RAE/100 g, on a dry weight basis.

Suitably, the nutritional composition comprises vitamin D in an amount of at least about 2.5 μg/100 g, at least about 5 μg/100 g, at least about 10 μg/100 g, or at least about 15 μg/100 g, on a dry weight basis. Suitably, the nutritional composition comprises vitamin D in an amount of about 100 μg/100 g or less, about 75 μg/100 g or less, or about 50 μg/100 g or less, on a dry weight basis. Suitably, the nutritional composition comprises vitamin D in an amount of from about 2.5 μg/100 g to about 100 μg/100 g, from about 5 μg/100 g to about 100 μg/100 g, from about 10 μg/100 g to about 75 μg/100 g or from about 15 μg/100 g to about 50 μg/100 g, on a dry weight basis.

### Oligosaccharides

A nutritional composition (e.g. milk formula) according to the invention may contain any suitable amounts of oligosaccharides.

Suitably, the nutritional composition comprises one or more oligosaccharide in a total amount of at least about 0.5 wt %, at least about 1 wt %, or at least about 2 wt %, on a dry weight basis. Suitably, the nutritional composition comprises one or more oligosaccharide in a total amount of about 10 wt % or less, about 8 wt % or less or about 5 wt % or less, on a dry weight basis. Suitably, the nutritional composition comprises one or more oligosaccharide in a total amount of from about 0.5 wt % to about 10 wt %, about 1 wt % to about 8 wt %, or about 2 wt % to about 5 wt %, on a dry weight basis.

Suitably, the nutritional composition comprises at least one fucosylated oligosaccharide in an amount of about 0.05 wt % to about 3 wt %, about 0.1 wt % to about 2 wt %, or about 0.2 wt % to about 1.5 wt %, on a dry weight basis. Suitably, the nutritional composition comprises at least one sialylated oligosaccharide in an amount of about 0.01 wt % to about 2 wt %, about 0.05 wt % to about 1.5 wt %, or about 0.07 wt % to about 1 wt %, on a dry weight basis. Suitably, the nutritional composition comprises at least one N-acetylated oligosaccharide in an amount of about 0.01 wt % to about 1 wt %, about 0.03 wt % to about 0.6 wt %, or about 0.05 wt % to about 0.5 wt %, on a dry weight basis.

Suitably, the nutritional composition comprises 2′FL in an amount of at least about 0.5 wt %, at least about 0.8 wt %, or at least about 1 wt %, on a dry weight basis. Suitably, the nutritional composition comprises 2′FL in an amount of about 5 wt % or less, about 4 wt % or less or about 3 wt % or less, on a dry weight basis. Suitably, the nutritional composition comprises 2′FL in an amount of from about 0.5 wt % to about 5 wt %, about 0.8 wt % to about 4 wt %, or about 1 wt % to about 3 wt %, on a dry weight basis.

Suitably, the nutritional composition comprises diFL in an amount of at least about 0.1 wt %, at least about 0.15 wt %, or at least about 0.2 wt %, on a dry weight basis. Suitably, the nutritional composition comprises diFL in an amount of about 1 wt % or less, about 0.8 wt % or less or about 0.6 wt % or less, on a dry weight basis. Suitably, the nutritional composition comprises diFL in an amount of from about 0.1 wt % to about 1 wt %, about 0.15 wt % to about 0.8 wt %, or about 0.2 wt % to about 0.6 wt %, on a dry weight basis.

Suitably, the nutritional composition comprises 6′-SL in an amount of at least about 0.1 wt %, at least about 0.15 wt %, or at least about 0.2 wt %, on a dry weight basis. Suitably, the nutritional composition comprises 6′-SL in an amount of about 1 wt % or less, about 0.8 wt % or less or about 0.6 wt % or less, on a dry weight basis. Suitably, the nutritional composition comprises 6′-SL in an amount of from about 0.1 wt % to about 1 wt %, about 0.15 wt % to about 0.8 wt %, or about 0.2 wt % to about 0.6 wt %, on a dry weight basis.

Suitably, the nutritional composition comprises 3′-SL in an amount of at least about 0.1 wt %, at least about 0.15 wt %, or at least about 0.2 wt %, on a dry weight basis. Suitably, the nutritional composition comprises 3′-SL in an amount of about 1 wt % or less, about 0.8 wt % or less or about 0.6 wt % or less, on a dry weight basis. Suitably, the nutritional composition comprises 3′-SL in an amount of from about 0.1 wt % to about 1 wt %, about 0.15 wt % to about 0.8 wt %, or about 0.2 wt % to about 0.6 wt %, on a dry weight basis.

Suitably, the nutritional composition comprises LNT in an amount of at least about 0.2 wt %, at least about 0.3 wt %, or at least about 0.4 wt %, on a dry weight basis. Suitably, the nutritional composition comprises LNT in an amount of about 2.5 wt % or less, about 2 wt % or less or about 1.5 wt % or less, on a dry weight basis. Suitably, the nutritional composition comprises LNT in an amount of from about 0.2 wt % to about 2.5 wt %, about 0.3 wt % to about 2 wt %, or about 0.4 wt % to about 1.5 wt %, on a dry weight basis.

Suitably, the nutritional composition comprises LNnT in an amount of at least about 0.2 wt %, at least about 0.3 wt %, or at least about 0.4 wt %, on a dry weight basis. Suitably, the nutritional composition comprises LNnT in an amount of about 2.5 wt % or less, about 2 wt % or less or about 1.5 wt % or less, on a dry weight basis. Suitably, the nutritional composition comprises LNnT in an amount of from about 0.2 wt % to about 2.5 wt %, about 0.3 wt % to about 2 wt %, or about 0.4 wt % to about 1.5 wt %, on a dry weight basis.

Suitably, the nutritional composition comprises 2′FL in a total amount of from about 0.05 g/L to about 2 g/L, from about 0.1 g/L to about 1 g/L, from about 0.15 g/L to about 0.8 g/L, from about 0.2 g/L to about 0.7 g/L, or from about 0.25 g/L to about 0.6 g/L (the concentration may refer to the concentration after the composition has been reconstituted e.g. with water).

Suitably, the nutritional composition comprises fucosylated oligosaccharide(s) (e.g. 2′FL and/or diFL) in a total amount of from about 0.1 g/L to about 4 g/L, from about 0.1 g/L to about 3.5 g/L, from about 0.15 g/L to about 3 g/L, from about 0.2 g/L to about 2.5 g/L, from about 0.3 g/L to about 2 g/L, from about 0.4 g/L to about 2 g/L, or from about 0.5 g/L to about 2 g/L (the concentration may refer to the concentration after the composition has been reconstituted e.g. with water). In a particular embodiment, the nutritional composition comprises from about 0.2 g/L to about 1.8 g/L of total fucosylated oligosaccharide(s).

Suitably, the nutritional composition comprises sialylated oligosaccharide(s) (e.g. 3′-sialyllactose (3′-SL) and/or 6′-sialyllactose (6′-SL) in a total amount of from about 0.05 g/L to about 0.75 g/L, from about 0.05 g/L to about 0.5 g/L, from about 0.1 g/L to about 0.3 g/L, or from about 0.1 g/L to about 0.4 g/L (the concentration may refer to the concentration after the composition has been reconstituted e.g. with water). In a particular embodiment, the nutritional composition comprises from about 0.12 g/L to about 0.4 g/L of total sialylated oligosaccharide(s).

Suitably, the nutritional composition comprises N-acetylated oligosaccharide(s) (e.g. LNT and/or LNnT) in a total amount of from about 0.05 g/L to about 0.5 g/L, from about 0.1 g/L to about 0.5 g/L, from about 0.2 g/L to about 0.4 g/L, or about 0.3 g/L (the concentration may refer to the concentration after the composition has been reconstituted e.g. with water).

Suitably, the nutritional composition comprises:
at least one sialylated oligosaccharide in in a total amount of from about 0.05 g/L to about 0.75 g/L, from about 0.05 g/L to about 0.5 g/L, from about 0.1 g/L to about 0.3 g/L, or from about 0.1 g/L to about 0.4 mg/L;
at least one fucosylated oligosaccharide in a total amount of from about 0.1 g/L to about 4 g/L, from about 0.1 g/L to about 3.5 g/L, from about 0.15 to about 3 g/L, from about 0.2 g/L to about 2.5 g/L, from about 0.3 g/L to about 2 g/L, from about 0.4 g/L to about 2 g/L, or from about 0.5 g/L to about 2 g/L; and/or
at least one N-acetylated oligosaccharide in a total amount of from about 0.05 g/L to about 0.5 g/L, from about 0.1 g/L to about 0.5 g/L, or from about 0.2 g/L to about 0.4 g/L

### Probiotics

A nutritional composition (e.g. milk formula) according to the invention may contain any suitable amounts of probiotic.

Suitably, the nutritional composition comprises the one or more probiotic in a total amount of at least about 10⁵ cfu/100 g, at least about 10⁶ cfu/100 g, at least about 10⁷ cfu/100 g, or at least about 10⁸ cfu/100 g, at least about 10⁹ cfu/100 g, or at least about 10¹⁰ cfu/100 g on a dry weight basis. Suitably, the nutritional composition comprises the one or more probiotic in a total amount of about 10¹² cfu/100 g or less, about 10¹¹ cfu/100 g or less, about 10¹⁰ cfu/100 g or less, on a dry weight basis. Suitably, the nutritional composition comprises the one or more probiotic in a total amount of about 10⁶ cfu/100 g to about 10¹² cfu/100 g, about 10⁷ cfu/100 g to about 10¹¹ cfu/100 g, or about 10⁸ cfu/100 g to about 10¹⁰ cfu/100 g, on a dry weight basis.

Suitably, the nutritional composition comprises Bifidobacterium longum (e.g. B. infantis) in an amount of at least about 10⁵ cfu/100 g, at least about 10⁶ cfu/100 g, at least about 10⁷ cfu/100 g, or at least about 10⁸ cfu/100 g, at least about 10⁹ cfu/100 g, or at least about 10¹⁰ cfu/100 g on a dry weight basis. Suitably, the nutritional composition comprises Bifidobacterium longum (e.g. B. infantis) in an amount of about 10¹² cfu/100 g or less, about 10¹¹ cfu/100 g or less, about 10¹⁰ cfu/100 g or less, on a dry weight basis. Suitably, the nutritional composition comprises Bifidobacterium longum (e.g. B. infantis) in an amount of about 10⁶ cfu/100 g to about 10¹² cfu/100 g, about 10⁷ cfu/100 g to about 10¹¹ cfu/100 g, or about 10⁸ cfu/100 g to about 10¹⁰ cfu/100 g, on a dry weight basis.

Suitably, the nutritional composition comprises Bifidobacterium animalis (e.g. B. lactis) in an amount of at least about 10⁵ cfu/100 g, at least about 10⁶ cfu/100 g, at least about 10⁷ cfu/100 g, or at least about 10⁸ cfu/100 g, at least about 10⁹ cfu/100 g, or at least about 10¹⁰ cfu/100 g on a dry weight basis. Suitably, the nutritional composition comprises Bifidobacterium animalis (e.g. B. lactis) in an amount of about 10¹² cfu/100 g or less, about 10¹¹ cfu/100 g or less, about 10¹⁰ cfu/100 g or less, on a dry weight basis. Suitably, the nutritional composition comprises Bifidobacterium animalis (e.g. B. lactis) in an amount of about 10⁶ cfu/100 g to about 10¹² cfu/100 g, about 10⁷ cfu/100 g to about 10¹¹ cfu/100 g, or about 10⁸ cfu/100 g to about 10¹⁰ cfu/100 g, on a dry weight basis.

Suitably, the nutritional composition comprises Lactobacillus rhamnosus in an amount of at least about 10⁵ cfu/100 g, at least about 10⁶ cfu/100 g, at least about 10⁷ cfu/100 g, or at least about 10⁸ cfu/100 g, at least about 10⁹ cfu/100 g, or at least about 10¹⁰ cfu/100 g on a dry weight basis. Suitably, the nutritional composition comprises Lactobacillus rhamnosus in an amount of about 10¹² cfu/100 g or less, about 10¹¹ cfu/100 g or less, about 10¹⁰ cfu/100 g or less, on a dry weight basis. Suitably, the nutritional composition comprises Lactobacillus rhamnosus in an amount of about 10⁶ cfu/100 g to about 10¹² cfu/100 g, about 10⁷ cfu/100 g to about 10¹¹ cfu/100 g, or about 10⁸ cfu/100 g to about 10¹⁰ cfu/100 g, on a dry weight basis.

### Protein

A nutritional composition (e.g. milk formula) according to the invention may contain a protein source. This is particularly preferable in the case where a nutritional composition of the invention is a milk formula. The protein may be in an amount of from about 1 g to about 4 g per 100 kcal, or about 1.5 g to about 3 g per 100 kcal.

Protein sources based on, for example, whey, casein and mixtures thereof may be used as well as plant based protein sources, for example, based on soy. As far as whey proteins are concerned, the protein source may be based on acid whey or sweet whey or mixtures thereof and may include alpha-lactalbumin and beta-lactoglobulin in any desired proportions. In some embodiments the protein source is whey predominant (i.e. more than 50% of proteins are coming from whey proteins, such as 60%> or 70%>). The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10%> by weight of lysine; for example about 7% by weight of lysine which greatly improves the nutritional quality of the protein source.

In one particular embodiment, the proteins of the composition are hydrolysed, fully hydrolysed or partially hydrolysed. The degree of hydrolysis (DH) of the protein can be between 2 and 20, or between 8 and 40, or between 20 and 60 or between 20 and 80 or more than 10, 20, 40, 60, 80 or 90.

At least 70%, 80%, 85%, 90%, 95% or 97% of the proteins may be hydrolysed. In a particular embodiment, 100% of the proteins are hydrolysed.

In one particular embodiment, the proteins of the composition are plant based protein.

### Carbohydrate

A nutritional composition (e.g. milk formula) according to the present invention may contain a carbohydrate source. This is particularly preferable in the case where a nutritional composition of the invention is a milk formula. The carbohydrate may be in an amount of from about 5 g to about 20 g per 100 kcal, or about 10 g to about 15 g per 100 kcal.

Any carbohydrate source conventionally found in milk formula such as lactose, sucrose, saccharose, maltodextrin, starch and mixtures thereof may be used although one of the preferred sources of carbohydrates for milk formula is lactose.

### Lipid

A nutritional composition (e.g. milk formula) according to the present invention may contain lipids and essential fatty acids. This is particularly preferable in the case where a nutritional composition of the invention is a milk formula. The lipids may be in an amount of from about 1 g to about 10 g per 100 kcal, or about 2 g to about 6 g per 100 kcal.

Non-limiting examples of lipids include: palm olein, high oleic sunflower oil, high oleic safflower oil, canola oil, fish oil, coconut oil, bovine milk fat, and combinations thereof. It may be particularly beneficial if the composition comprises fat in an amount of about 25 to about 30 g/100 g dry weight of the composition. Non-limiting examples of essential fatty acids include: linoleic acid (LA), α-linolenic acid (ALA). Compositions of the invention may further contain gangliosides monosialoganglioside-3 (GM3) and disialogangliosides 3 (GD3), and combinations thereof.

### Other Components

A nutritional composition (e.g. milk formula) of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin B1, vitamin B2, vitamin B3, vitamin B6, vitamin B12, vitamin E, vitamin K1, vitamin C, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population. If necessary, a nutritional composition of the invention may contain emulsifiers and stabilisers such as soy, lecithin, citric acid esters of mono- and diglycerides, and the like.

A nutritional composition (e.g. milk formula) of the invention may also contain other substances which may have a beneficial effect, especially on bone health or bone development, such as lactoferrin, osteopontin, TGFbeta, slgA, glutamine, nucleotides, nucleosides, and the like.

### Preparation of Compositions

The compositions according to the present invention may be prepared by any known or otherwise suitable manner. For example, a nutritional composition, e.g. a milk formula, may be proposed by blending together a source of protein with a carbohydrate source and a lipid source in appropriate proportions. If used, emulsifiers may be included at this stage. Vitamins and minerals may be added at this stage, but may also be added later to avoid thermal degradation. Water, preferably water which has been subjected to reverse osmosis or deionized water, may then be added and mixed in to form a liquid mixture. The temperature of mixing is preferably room temperature, but may also be higher. The liquid mixture may then be thermally treated to reduce bacterial loads. The mixture may then be homogenized.

If it is desired to produce a powdered composition, the homogenized mixture is dried in a suitable drying apparatus, such as a spray drier or freeze drier and converted into powder.

Processes used in the manufacture of formula are based on the concept that the products must be nutritionally adequate and microbiologically safe to consume. Thus, steps that eliminate or restrict microbiological growth are central to production processes. The processing technology for each specific formula is proprietary to the manufacturer but, in general, it involves the preservation of an oil-in-water (o/w) emulsion by dehydration in the case of powder products or, sterilization in the case of ready-to-feed or concentrated liquid products. Powdered formula may be produced using various processes, such as dry blending dehydrated ingredients to constitute a uniform formula or hydrating and wet-mixing a mixture of macro-ingredients, such as fat, protein and carbohydrate ingredients and then evaporating and spray drying the resultant mixture. A combination of the two processes described above may be used where a base powder is first produced by wet-mixing and spray drying all or some of the macro-ingredients and then dry blending the remaining ingredients, including carbohydrate, minerals and vitamins and other micronutrients, to create a final formula. Liquid formulae are available in a ready-to-feed format or as a concentrated liquid, which requires dilution, normally 1:1, with water. The manufacturing processes used for these products are similar to those used in the manufacture of recombined milk.

If it is desired to produce a liquid formula, the homogenized mixture may be filled into suitable containers, preferably aseptically. However, the liquid composition may also be retorted in the container, suitable apparatus for carrying out the filling and retorting of this nature is commercially available.

### Subject

The subject may be any suitable subject. Suitably, the subject may be a mammal. In preferred embodiments, the subject is a human. In other embodiments, the subject is an animal, preferably wherein the animal is a pet. A pet may be an animal selected from dogs, cats, birds, fish, rodents such as mice, rats, and guinea pigs, rabbits, etc. In some embodiments, the pet is a small dog breed.

In some embodiments, the subject is a juvenile, an adolescent, a child. The term “juvenile” may refer to an individual that has not yet reached adulthood. The term “adolescent” may refer to an individual during the period from the onset of puberty to adulthood. The term “child” may refer an individual between the stages of birth and puberty.

In preferred embodiments, the subject is about 3 years of age or older. In some embodiments, the subject is about 4 years of age or older or about 5 years of age or older.

In preferred embodiments, the subject is about 10 years of age or younger. In some embodiments, the subject is about 9 years of age or younger, about 8 years of age or younger, about 7 years of age or younger, about 6 years of age or younger, or about 5 years of age or younger.

In some embodiments, the subject is about 3 years to about 10 years of age, about 3 years to about 9 years of age, about 3 years to about 8 years of age, about 3 years to about 7 years of age, about 3 years to about 6 years of age, or about 3 years to about 5 years of age.

The present invention is particularly suitable for children who were born preterm or with low-birth weight or experienced intra-uterine growth retardation or who suffered from growth stunting because of malnutrition or experienced disease such as Crohn's disease and/or celiac disease and/or cancer or who were treated with drugs leading to malabsorption, anorexia and/or metabolic bone disease, such as chemotherapy drugs and/or corticosteroids. The present invention is particularly preferred for use in children who were born preterm or with low-birth weight or experienced intra-uterine growth retardation, or with intra-uterine malnutrition or who suffered growth delay. The present invention is also suitable for children at risk of bone disease, having a family history of bone disease, or having already experienced at least one, preferably several, episode(s) of fracture.

In some embodiments, the subject suffered from and/or is suffering from stunted growth. The definition of stunting may refer to the “height for age” value to be less than two standard deviations of the WHO Child Growth Standards median (see e.g. De Onis, M. and Branca, F., 2016. Maternal & child nutrition, 12, pp. 12-26).

In some embodiments, the subject suffered from and/or is suffering from faltering growth. The term “faltering growth” may describe a pattern of slower weight gain than expected for age and sex in children and other adolescents (see e.g. King, C. and Davis, T., 2010. European journal of clinical nutrition, 64 (1), pp. S11-S13). In some embodiments, the subject suffered from and/or is suffering from growth stunting and/or faltering growth because of malnutrition or experienced disease such as anorexia, Crohn's disease and/or celiac disease. In some embodiments, the subject suffered from and/or is suffering from growth stunting and/or faltering growth because of treatment with drugs leading to malabsorption, anorexia and/or metabolic bone disease, such as chemotherapy drugs and/or corticosteroids.

In some embodiments, the subject was born preterm or with low-birth weight or experienced intra-uterine growth retardation. The term “preterm infant” may refer to an infant born at least than 37 weeks gestational age. The term “low birth weight infant” may refer to an infant having a live-born weight less than 2,500 g.

Methods for Enhancing Bone Growth and/or Bone Strength

The present inventors have shown that the combination of the present invention may be used to enhance bone growth and/or bone strength in a subject.

Within the context of the present invention, the term “enhancing bone growth and/or bone strength” may refer to, in particular, one or more of the following physiological processes: bone catch-up growth, bone mass acquisition, optimization of peak bone mass, promotion of bone formation, promotion of bone anabolism, promotion of bone mineralization, increase of bone mineral density and micro-architecture, modulation of bone biomechanical properties, and modulation the ratio of bone formation and/or bone resorption.

In one aspect, the present invention provides a combination according to the present invention for use in enhancing bone growth and/or bone strength in a subject.

In one aspect, the present invention provides use of a combination according to the present invention in the manufacture of a medical food product for enhancing bone growth and/or bone strength in a subject.

In one aspect, the present invention provides a method for enhancing bone growth and/or bone strength in a subject, said method comprising administering a therapeutically effective amount of a combination according to the present invention to a subject in need thereof.

As used herein, “promoting bone growth and/or strength” may refer to the support of normal bone growth and/or strength, for example during childhood and adolescence. Supporting normal bone growth and/or strength may result in normal bone anatomy and physiology.

Suitable methods and parameters to determine bone growth and bone strength will be known to the skilled person (see e.g. Donnelly, E., 2011. Clinical Orthopaedics and Related Research, 469 (8), pp. 2128-2138). Suitably, normal bone growth and/or strength may be determined using one or more bone parameter selected from: trabecular bone volume fraction (BV/TV), bone mineral density (BMD), bone mineral content (BMC), cortical bone volume (Ct.BV), medio-lateral diameter, antero-posterior diameter, bone ultimate force (FMax), and bone stiffness. In some embodiments, normal bone growth and/or strength is determined using one or more bone parameter selected from: bone mineral density (BMD), trabecular bone volume fraction (BV/TV), cortical bone volume (Ct.BV), and bone ultimate force (FMax). Suitable methods to determine these parameters will be available to the skilled person.

### Methods for Promoting Catch-Up Growth

The combination of the present invention may promote catch-up growth e.g. in subjects with stunted growth and/or faltering growth.

In one aspect, the present invention provides a combination according to the present invention for use in promoting catch-up growth in a subject.

In one aspect, the present invention provides use of a combination according to the present invention in the manufacture of a medical food product for promoting catch-up growth in a subject.

In one aspect, the present invention provides a method for promoting catch-up growth in a subject, said method comprising administering a therapeutically effective amount of a combination according to the present invention to a subject in need thereof.

As used herein, “catch-up growth” may refer to height velocity above the limits of normal for age for at least 1 year after a transient period of growth inhibition and may be complete or incomplete (see e.g. Wit, J. M. and Boersma, B., 2002. Journal of Pediatric Endocrinology and Metabolism, 15, pp. 1229-1242.

Suitable method and parameters to determine catch-up growth will be known to the skilled person. Suitably, catch-up growth may be determined using height velocity or height standard deviation score (see e.g. Frongillo, E. A., Leroy, J. L. and Lapping, K., 2019. Advances in Nutrition, 10 (3), pp. 372-379 and Desmond, C. and Casale, D., 2017. PloS one, 12 (12), p.e0189135).

In some embodiments, the catch-up growth is determined in absolute terms of linear growth (i.e. the height deficit from the healthy reference population mean is reduced). In some embodiments, the catch-up growth is determined in relative terms of linear growth (i.e. the height-for-age z-score is improved and/or passes the −2SD or −1SD cut-off points).

### EXAMPLES

The invention is further described with reference to the following examples. It will be appreciated that the invention as claimed is not intended to be limited in any way by these examples.

### Example 1—Effect of Combination of Vitamins K2, A, D and HMO Mixture on Bone Development

### Results

The effect of vitamins K2, A, D and short-chain fatty acids (SCFA) on osteoblast alkaline phosphatase (ALP) activity and osteocalcin mRNA levels in a pre-osteoblastic cell line was investigated.

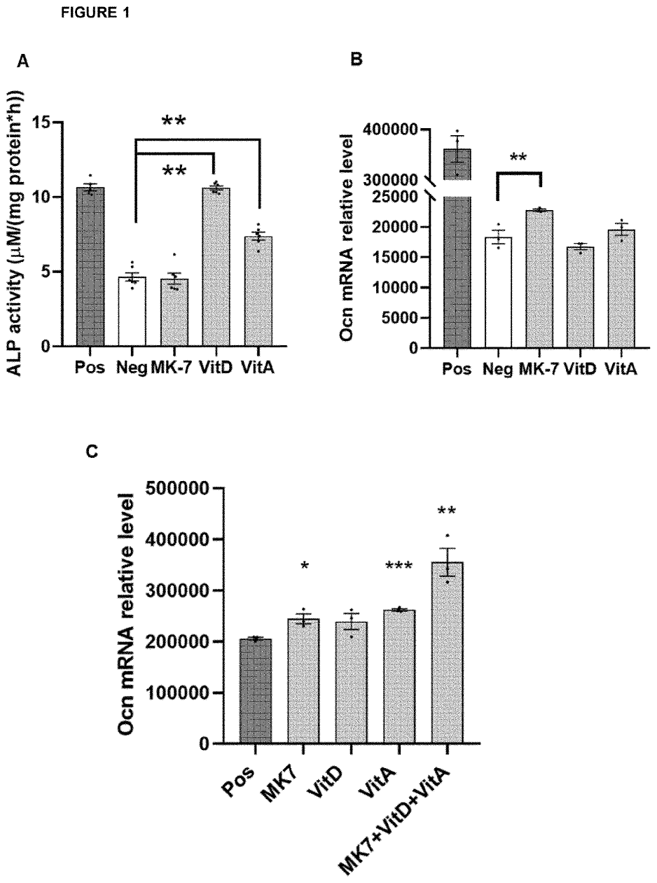

After 7 days of differentiation (without ascorbic acid), vitamin A and vitamin D showed a significant effect on ALP activity, which is a marker of osteoblast differentiation, however vitamin K2 showed no significant effect (see FIG. 1A). On the other hand, after 21 days of differentiation (without ascorbic acid), vitamin K2 showed a significant effect on osteocalcin expression, which is a key protein for osteoblast mineralization activity, however, vitamin A and vitamin D showed no significant effect (see FIG. 1B).

After 28 days of differentiation (with ascorbic acid), the combination of vitamins K2, A, and D synergistically promoted osteoblast mineralization (see FIG. 1C).

The effect of SCFAs was further investigated. Three mixes of 75% acetate, 20% propionate, 5% butyrate were evaluated to mimic normal physiological conditions (20 μM SCFAs), physiological conditions following prebiotic supplementation (50 μM SCFAs), and physiological conditions following synbiotic supplementation (60 μM SCFAs).

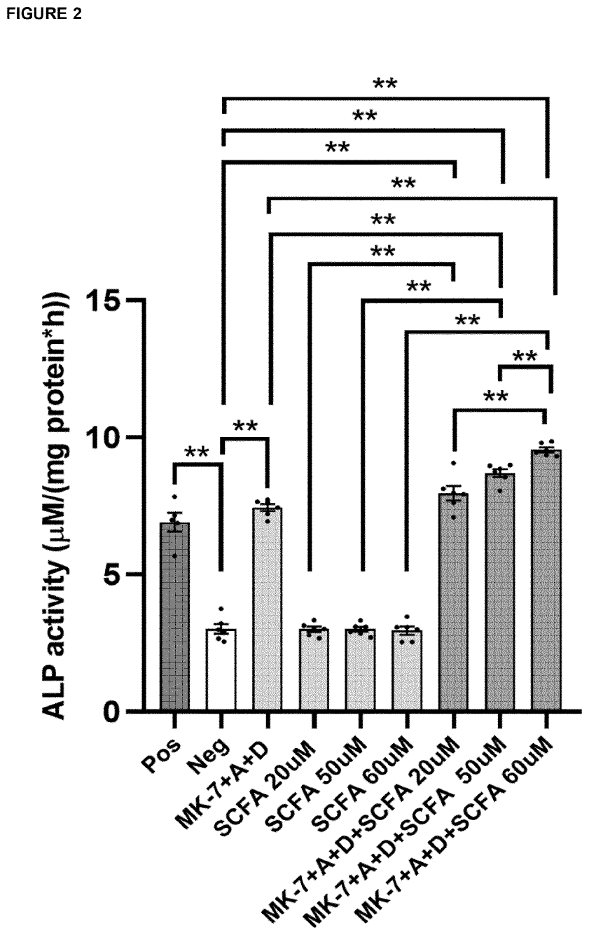

After 7 days of differentiation (without ascorbic acid), the combination of vitamins K2, A, and D with 50 μM or 60 μM SCFAs synergistically promoted osteoblast differentiation (see FIG. 2). This effect was up to 28.4% greater than the additive effect.

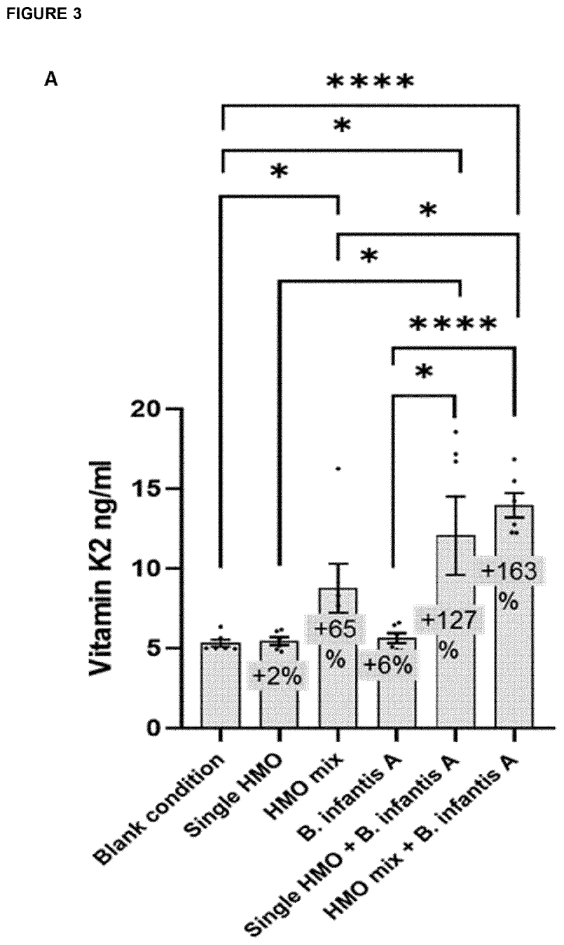

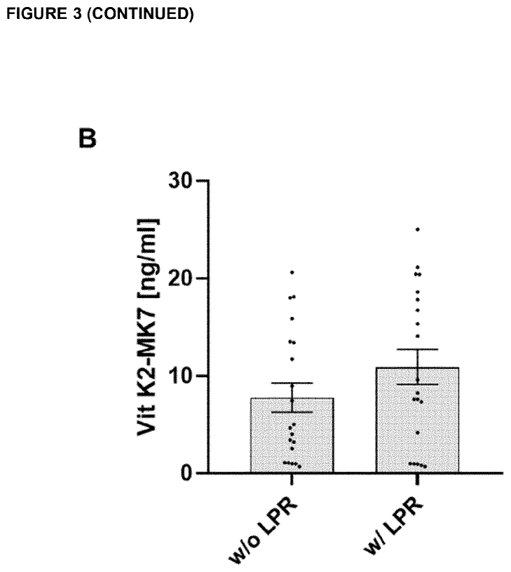

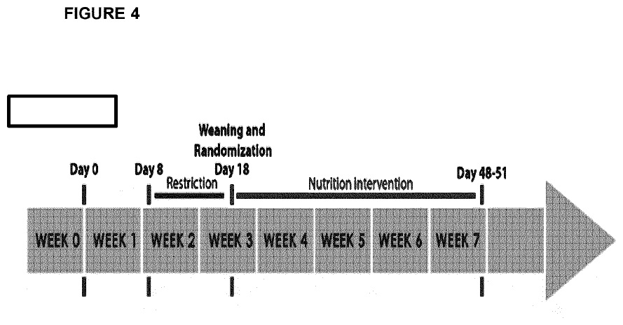

In a gut model that mimics the gastrointestinal tract, it was further shown that a mixture of human milk oligosaccharides (2.5 g/L 2FL, DiFL, LNnT, LNT, and 6SL) can promote vitamin K2 production in the gastrointestinal tract and that the addition of B. infantis can enhance this effect (see FIG. 3A). It was also shown that in a milk matrix, the addition of Lactobacillus rhamnosus LPR (4.5×10⁷ cfu/ml) can further increase vitamin K2 production in the gastrointestinal tract by about 40% (see FIG. 4).

### Material and Methods

### MC3T3-E1 Subclone 4 Culture and Treatments Conditions

The pre-osteoblastic cell line MC3T3-E1 subclone 4 (CRL-2593) was purchased from ATCC (Manassas; Virginia, USA). Cells were maintained in growth medium (GM) composed of ascorbic acid-free αMEM (ThermoFisher Scientific), supplemented with 10% fetal calf serum (FCS, ThermoFisher Scientific) and 1% penicillin/streptomycin. All culture media were refreshed every 2-3 days. Cells were passaged with trypsin/EDTA solution at less than 80% confluence. To induce differentiation into osteoblasts, cells were seeded at 5×10⁴ cells/cm² and grown to confluency in GM for 24 h. Then, the medium was switched to differentiation medium (DM) composed of GM supplemented with 10 mM β-glycerophosphate and treatment solutions of interest. Ascorbic acid 50 μg/ml was also added to the DM depending on the experiment.

### Osteoblast Alkaline Phosphatase Activity

MC3T3-E1 subclone 4 cells were differentiated for 7 days without ascorbic acid, except for the positive control, and with the following treatment solutions: a mix composed of 3 μM of vit. K2 (menaquinone-7), 100 nM of vit. A (all-trans-retinoic acid) and 1 nM of vit. D (1α,25-Dihydroxyvitamin D3); a mix called “SCFA20” composed of 15 μM of sodium acetate, 4 μM of sodium propionate and 1 μM of sodium butyrate; a mix called “SCFA50” composed of 37.5 μM of sodium acetate, 10 μM of sodium propionate and 2.5 μM of sodium butyrate; a mix called “SCFA60” composed of 45 μM of sodium acetate, 12 μM of sodium propionate and 3 μM of sodium butyrate. The vitamin mix was also combined to the 3 different combinations of short-chain fatty acids (SCFA).

After 7 days of differentiation, cells were then collected for alkaline phosphatase (ALP) activity measurement as described previously with minor modifications. Briefly, cells were lysed by heat shock and collected in ALP buffer (1 M diethanolamine, 0.24 M MgCl₂, pH 9.8). Enzymatic reaction was monitored at 405 nm after 4-Nitrophenyl phosphate disodium salt hexahydrate addition. Michaelis-Menten kinetics were evaluated at 30° C. for 30 min. Vₘₐₓ was used as a proxy for ALP activity. The activity value was normalized by protein content measured via the Pierce BCA Protein Assay Kit (ThermoFisher Scientific) according to the manufacturer's instructions.

### Osteocalcin RNA Extraction

MC3T3-E1 subclone 4 cells were differentiated for 21 days (in DM without ascorbic acid except for the positive control) or 28 days (in DM containing ascorbic acid). Cells were then collected for gene expression analysis. RNA was extracted using the RNeasy plus mini kit (Qiagen; Hilden, Germany) with the QIAcube (Qiagen,) according to the manufacturer's instructions. Briefly, cells were lysed in RLT buffer and spun in a QIAshredder column (Qiagen) before being processed by the QIAcube. RNA concentrations were measured using the DropSense96 (TRINEAN, Gentbrugge, Belgium).

Reverse Transcription and Quantitative PCR (qPCR)

cDNAs were synthetized using High-Capacity cDNA Reverse Transcription Kit (AppliedBiosystems; Waltham, Massachusetts, USA) according to the manufacturer's instructions. Briefly, 0.7 μg of RNA were mixed to the components of the kit and were reverse transcribed with the following program: 10 min at 25° C., 120 min at 37° C., and 5 min at 85° C. cDNAs were diluted 7× with RNase-free water and used for qPCR using the LightCycler 1536 DNA Green Master kit (Roche; Basel, Switzerland) according to the manufacturer's instructions. Briefly, cDNAs were diluted 7× in a solution containing the master mix, the Bright Green, and the DNA primers targeting osteocalcin (Ocn) and beta-2-microglobulin (B2m, housekeeping gene) the following with nucleotide sequences Ocn-f ACCATCTTTCTGCTCACTCTG, Ocn-r GTTCACTACCTTATTGCCCTCC, B2m-f CACTGACCGGCCTGTATGCT, B2m-r GTATGTTCGGCTTCCCATTCTC. Reaction ran in a LightCycler 480 II (Roche) with the following program: 7 min at 95° C., 40 cycles of 1 s at 95° C. and 30 s at 60° C. The relative gene expression was evaluated via the 2\hat{ }-ΔCt method.

### Prodigest and Vitamin K2 Production

To evaluate intestinal production of vitamin K2, a Simulator of the Human Microbial Ecosystem (SHIME®) was used (see Van de Wiele, T., et al., 2015. The Impact of Food Bioactives on Health: in vitro and ex vivo models, pp. 305-317). SHIME assays typically consist of a colonic fermentation of a selected dose of the test compound(s) under simulated conditions representative for the gastrointestinal tract of a subject. Production of menaquinone-7 was determined by supercritical fluid chromatography tandem mass spectrometry (SFC-MS/MS).

For the current set of experiments a two-stage batch system mimicking the upper gastro-intestinal tract (Upper GIT, stomach and small intestine) and colonic conditions were used as simplified SHIME® system. A cow milk-based infant beverage, also called infant formula-milk, containing minerals adapted for the age group was used in these studies.

In order to simulate the absorptive processes occurring in the small intestine of infants, a dialysis approach was applied by using a cellulose membrane with a cut-off of 14 kDa. By introducing the small intestinal suspension within a dialysis membrane, molecules such as digested amino acids, sugars, micronutrients and minerals were gradually removed from the upper gastro-intestinal matrices.

Furthermore, a gradual pH decrease during the stomach incubation going from 5.5 to 3.0 during 1 hour of incubation was implemented to simulate the gastric pH of infants. Also, during the first 30 minutes of small intestinal incubation (duodenum), a fixed pH of 4.5 was implemented to allow the available minerals to optimally absorb. In the following 145 minutes of the small intestinal phase (jejunum+ileum), a pH of 7 was introduced. The milk matrix after exposure to gastric and small intestinal conditions was transferred to the colonic compartment containing the fecal sample of an infant.

Fresh fecal material was collected from a 12-month-old infant donor. Fecal suspension was prepared and mixed with a protectant. At the start of the short-term colonic incubation, the test ingredients (Blank; Single HMO (1.3 g/L 2FL); HMO mix (2.5 g/L 2FL, DiFL, LNnT, LNT, and 6SL); B. infantis (10⁷ cfu/ml); Single HMO+B. infantis; HMO mix+B. infantis.) were added to sugar-depleted nutritional medium containing basal nutrients present in the colon (e.g., host-derived glycans such as mucin).

### Example 2

A preclinical experimental setup was followed as shown in FIG. 4.

Tibias were placed in separate histological cassette between foams and were fixed in 10% (v/v) neutral buffered formalin at room temperature for at least 48 h, decalcified for a minimum of 4 h in DC2 (if necessary), and then were embedded in paraffin until further histological processing. Two sagittal sections (5 μm) were cut with a microtome and stained according to a standard haematoxylin and eosin (H&E) protocol. A histomorphometry analysis of the resting, proliferative, pre-hypertrophic, and hypertrophic zones were performed to evaluate the total growth plate height as well as the height of each of the 4 precited zones (Guevara-Morales J M, Frohbergh M, Castro-Abril H, et al. Growth Plate Pathology in the Mucopolysaccharidosis Type VI Rat Model—An Experimental and Computational Approach. Diagnostics. 2020; 10 (6). doi: 10.3390/diagnostics10060360; Wilson K, Usami Y, Hogarth D, et al. Analysis of Association between Morphometric Parameters of Growth Plate and Bone Growth of Tibia in Mice and Humans. Cartilage. 2020; 13 (2_suppl): 315S-325S. doi: 10.1177/1947603519900800).

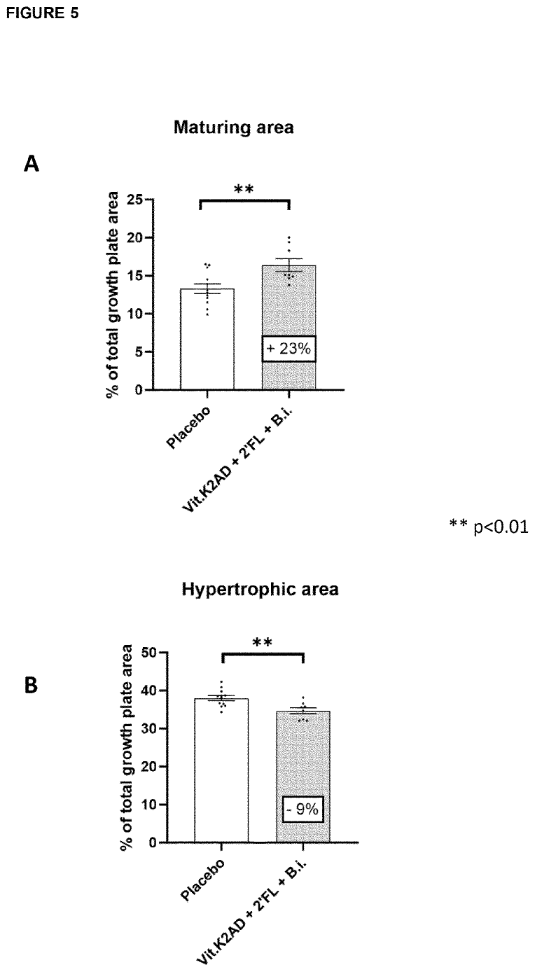

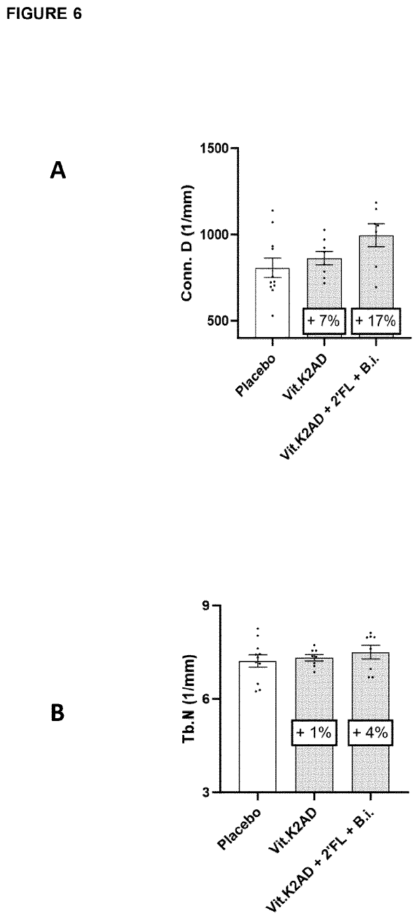

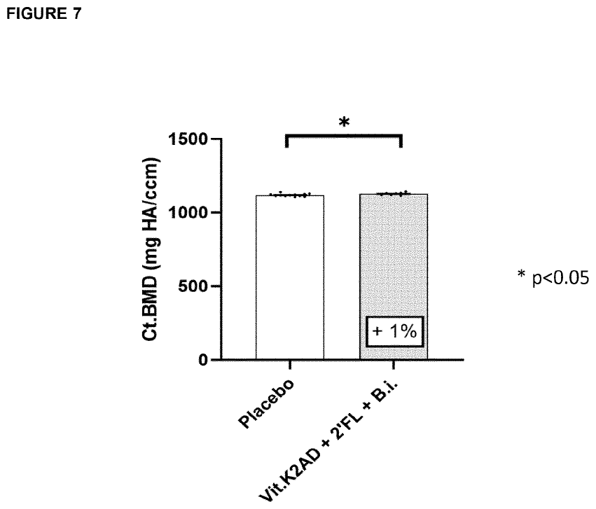

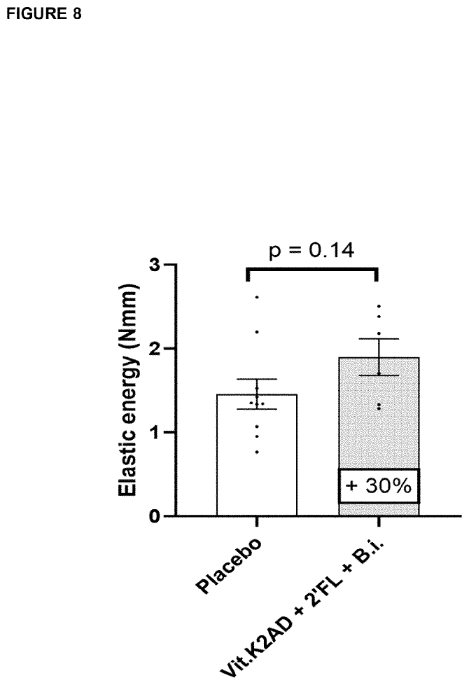

As can be seen in FIG. 5A&B, vitamin K2AD+synbiotic [2′-fucosyllactose+B. infantis] stimulated bone development through cartilage cells activation. The mature cartilage area was increased whereas hypertrophic cartilage area was decreased.

T

The impact of Vit. K2AD+/−synbiotic [2′-fucosyllactose+B. infantis] on trabecular connectivity density (Conn. D) and trabecular number (Tb.N) was assessed through micro-computed tomography on femur. Micro-computed tomography (μCT UCT35, Scanco Medical AG, Basserdorf, Switzerland) was used to assess trabecular microstructure at distal metaphysis of femurs, as previously described (N. Bonnet, J. Brun, J. C. Rousseau, L. T. Duong, S. L. Ferrari, Cathepsin K Controls Cortical Bone Formation by Degrading Periostin, J. Bone Miner. Res., 2017, 32 (7): 1432-1441). Briefly, trabecular bone region was evaluated using isotropic 6 μm voxels. To eliminate the primary spongiosa, 30 slices of bone under the distal growth plate were not considered. The 80 slices of secondary spongiosa directly below were analyzed. Morphometric variables were computed from binarized images using direct, three-dimensional techniques that do not rely on prior assumptions about the underlying structure (N. Bonnet, N. Laroche, L. Vico, E. Dolleans, D. Courteix, C. L. Benhamou, Assessment of trabecular bone microarchitecture by two different x-ray microcomputed tomographs: a comparative study of the rat distal tibia using Skyscan and Scanco devices, Med. Phys., 2009, 36 (4): 1286-97). The connectivity density (Conn. D) and the trabecular number (Tb.N) were assessed. As can be seen in FIG. 6A&B, Vit K2AD increased both femoral connectivity density (Conn. D) and trabecular number (Tb. N). The increase was higher when K2AD were combined with synbiotic [2′-fucosyllactose+B. infantis].
The impact of Vit. K2AD+synbiotic [2′-fucosyllactose+B. infantis] on cortical bone mineral density (Ct.BMD) was assessed through micro-computed tomography on femurs.

Micro-computed tomography (μCT UCT35, Scanco Medical AG, Basserdorf, Switzerland) was used to assess cortical microstructure at midshaft diaphysis of femurs, as previously described (N. Bonnet, J. Brun, J. C. Rousseau, L. T. Duong, S. L. Ferrari, Cathepsin K Controls Cortical Bone Formation by Degrading Periostin, J. Bone Miner. Res., 2017, 32 (7): 1432-1441). Briefly, cortical bone region was evaluated using isotropic 6 μm voxels. Femoral cortical structure was assessed using 60 continuous CT slides located at the femur midshaft. Morphometric variables were computed from binarized images using direct, three-dimensional techniques that do not rely on prior assumptions about the underlying structure (N. Bonnet, N. Laroche, L. Vico, E. Dolleans, D. Courteix, C. L. Benhamou, Assessment of trabecular bone microarchitecture by two different x-ray microcomputed tomographs: a comparative study of the rat distal tibia using Skyscan and Scanco devices, Med. Phys., 2009, 36 (4): 1286-97). Cortical bone mineral density (Ct.BMD) was assessed. As can be seen from FIG. 7, Vitamin K2AD+synbiotic [2′-fucosyllactose+B. infantis] increased femoral cortical bone mineral density (Ct. BMD).
the impact of Vitamin K2AD+synbiotic [2′-fucosyllactose+B. infantis] on femur strength was assessed through axial compression of the metaphysis.

1.7 mm high slices were cut from the femurs to isolate the metaphysis. The metaphysis slices were compressed axially to test the biomechanical properties of the femur as previously described (C. H. Turner, D. B. Burr, Basic biomechanical measurements of bone: a tutorial, Bone, 1993, 14 (4): 595-608). The load was applied in a compression mode at a nominal deformation rate of 2 mm/min until fracture. Load-displacement curves were recorded to determine the elastic energy. As can be seen in FIG. 8, Vitamin K2AD+synbiotic [2′-fucosyllactose+B. infantis] enhanced bone strength by increasing the elastic energy of the metaphyseal femur.

### EMBODIMENTS

Various preferred features and embodiments of the present invention will now be described with reference to the following numbered paragraphs (paras).
1. A combination of a vitamin mixture and a synbiotic for use in enhancing bone growth, strength and/or bone strength in a child or juvenile subject, wherein the vitamin mixture comprises or consists of vitamin K2, vitamin A, and vitamin D, and wherein the synbiotic comprises or consists of 2′-fucosyllactose (2′FL) and one or more probiotic.
2. The combination for use according to para 1, wherein vitamin K2 is administered to the subject in an amount of from about 5 μg/day to about 200 μg/day.
3. The combination for use according to para 1 or 2, wherein vitamin A is administered to the subject in an amount of from about 100 μg RE/day to about 1000 μg RE/day.
4. The combination for use according to any preceding para, wherein vitamin D is administered to the subject in an amount of from about 2.5 μg/day to about 100 μg/day.
5. The combination for use according to any preceding para, wherein the synbiotic comprises an oligosaccharide mixture comprising or consisting of 2′-fucosyllactose (2′FL) and at least one further oligosaccharide, preferably wherein the at least one further oligosaccharide comprises or consists of at least one sialylated oligosaccharide, at least one fucosylated oligosaccharide, and/or at least one N-acetylated oligosaccharide.
6. The combination for use according to para 5, wherein the at least one sialylated oligosaccharide is selected from the group consisting of 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof, preferably wherein the at least one sialylated oligosaccharide is selected from 3′-sialyllactose (3′-SL), 6′-sialyllactose (6′-SL) and combinations thereof.
7. The combination for use according to para 5 or 6, wherein the at least one fucosylated oligosaccharide is selected from the group consisting of 3-fucosyllactose (3FL), difucosyllactose (diFL), lacto-N-fucopentaose-I (LNFP-I), lacto-N-fucopentaose-II (LNFP-II), lacto-N-fucopentaose-III (LNFP-III), lacto-N-fucopentaose-V (LNFP-V), lacto-neofucopentaose V (LNnFP-V), lacto-N-difucosylhexaose-I (LNDFH-1), lacto-N-neodifucosylhexaose (LNnDFH), monofucosyllacto-n-hexaose-III (MFNLH-III), difucosyllacto-N-hexaose-a (DFLNHa) and combinations thereof, preferably wherein the at least one fucosylated oligosaccharide is difucosyllactose (diFL).
8. The combination for use according to any of paras 5 to 7, wherein the at least one N-acetylated oligosaccharide is selected from the group consisting of N-acetyl-glucosamine, N-acetyl-galactosamines, lacto-N-tetraose (LNT), lacto-N-neotetraose (LNnT), and combinations thereof, preferably wherein the at least one N-acetylated oligosaccharide is selected from lacto-N-tetraose (LNT), lacto-N-neotetraose (LNnT) and combinations thereof.
9. The combination for use according to any preceding para, wherein 2′FL is administered to the subject in an amount of from about 0.5 g/day to about 5 g/day.
10. The combination for use according to any preceding para, wherein the one or more probiotic comprises or consists of Bifidobacterium infantis.
11. The combination for use according to any preceding para, wherein B. infantis is administered to the subject in a total amount of from about 10⁶ cfu/day to about 10¹² cfu/day.
12. The combination for use according to any preceding para, wherein the synbiotic comprises a probiotic mixture comprising B. infantis and at least one further probiotic, preferably wherein the at least one further probiotic comprises or consists of Bifidobacterium animalis and/or Lactobacillus rhamnosus.
13. The combination for use according to any preceding para, wherein the combination is provided in the form of a nutritional composition.
14. The combination for use according to any preceding para, wherein the combination is provided in the form of a medical food product for clinical nutrition.
15. The combination for use according to any preceding para, wherein the combination is provided in the form of a milk formula.
16. The combination for use according to any of paras 13 to 15, wherein the composition comprises vitamin K2 in an amount of from about 5 μg/100 g to about 200 μg/100 g, on a dry weight basis.
17. The combination for use according to any of paras 13 to 16, wherein the composition comprises vitamin A in an amount of from about 100 μg RE/100 g to about 1000 μg RE/100 g, on a dry weight basis.
18. The combination for use according to any of paras 13 to 17, wherein the composition comprises vitamin D in an amount of from about 2.5 μg/100 g to about 100 μg/100 g, on a dry weight basis.
19. The combination for use according to any of paras 13 to 18, wherein the composition comprises 2′FL in an amount of from about 0.5 wt % to about 5 wt %, on a dry weight basis.
20. The combination for use according to any of paras 13 to 19, wherein the composition comprises B. infantis in an amount of from about 10⁶ cfu/100 g to about 10¹² cfu/100 g, on a dry weight basis.
21. The combination for use according to any preceding para, wherein the subject is human.
22. The combination for use according to any preceding para, wherein the subject is about 3 years of age or older and/or about 10 years of age or younger, preferably wherein the subject is from about 3 years to about 10 years of age.
23. The combination for use according to any of paras 1 to 20, wherein the subject is an animal, preferably wherein the animal is a pet.
24. The combination for use according to any preceding para, wherein the subject suffered from and/or is suffering from stunted growth and/or faltering growth.
25. The combination for use according to any preceding para, wherein the subject was born preterm or with low-birth weight or experienced intra-uterine growth retardation.
26. The combination for use according to any preceding para, wherein the combination is administered by oral administration.
27. The combination for use according to any preceding para, wherein the vitamin mixture and synbiotic are administered separately, simultaneously or sequentially, preferably wherein the vitamin mixture and synbiotic are administered simultaneously.
28. The combination for use according to any preceding para, wherein the combination synergistically enhances bone growth and/or bone strength.
29. The combination for use according to any preceding para, wherein the combination enhances bone mineralization.
30. The combination for use according to any preceding para, wherein the combination promotes osteoblast mineralization and/or osteoblast differentiation.
31. The combination for use according to any preceding para, wherein the combination improves one or more bone parameter selected from: bone mineral density (BMD), trabecular bone volume fraction (BV/TV), cortical bone volume (Ct.BV) and bone ultimate force (FMax).
32. The combination for use according to any preceding para, wherein the combination promotes catch-up growth, preferably wherein catch-up growth is determined using height velocity.
33. Use of a combination of a vitamin mixture and a synbiotic in the manufacture of a medical food product for enhancing bone growth and/or bone strength in a child or juvenile subject, wherein the vitamin mixture comprises or consists of vitamin K2, vitamin A, and vitamin D, and wherein the synbiotic comprises or consists of 2′-fucosyllactose (2′FL) and one or more probiotic.
34. A method for enhancing bone growth and/or bone strength in a child or juvenile subject, the method comprising administering to the subject an effective amount of a combination of a vitamin mixture and a synbiotic, wherein the vitamin mixture comprises or consists of vitamin K2, vitamin A, and vitamin D, and wherein the synbiotic comprises or consists of 2′-fucosyllactose (2′FL) and one or more probiotic.
35. Use of a synbiotic to promote vitamin K2 production in the gut of a subject.
36. Use according to para 35, wherein the synbiotic comprises an oligosaccharide mixture comprising or consisting of 2′-fucosyllactose (2′FL) and at least one further oligosaccharide, preferably wherein the at least one further oligosaccharide comprises or consists of at least one sialylated oligosaccharide, at least one fucosylated oligosaccharide, and/or at least one N-acetylated oligosaccharide.
37. Use according to para 36, wherein the at least one sialylated oligosaccharide is selected from the group consisting of 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof, preferably wherein the at least one sialylated oligosaccharide is selected from 3′-sialyllactose (3′-SL), 6′-sialyllactose (6′-SL) and combinations thereof.
38. Use according to para 36 or 37, wherein the at least one fucosylated oligosaccharide is selected from the group consisting of 3-fucosyllactose (3FL), difucosyllactose (diFL), lacto-N-fucopentaose-I (LNFP-I), lacto-N-fucopentaose-II (LNFP-II), lacto-N-fucopentaose-III (LNFP-III), lacto-N-fucopentaose-V (LNFP-V), lacto-neofucopentaose V (LNnFP-V), lacto-N-difucosylhexaose-I (LNDFH-1), lacto-N-neodifucosylhexaose (LNnDFH), monofucosyllacto-n-hexaose-III (MFNLH-III), difucosyllacto-N-hexaose-a (DFLNHa) and combinations thereof, preferably wherein the at least one fucosylated oligosaccharide is difucosyllactose (diFL).
39. Use according to any of paras 36 to 38, wherein the at least one N-acetylated oligosaccharide is selected from the group consisting of N-acetyl-glucosamine, N-acetyl-galactosamines, lacto-N-tetraose (LNT), lacto-N-neotetraose (LNnT), and combinations thereof, preferably wherein the at least one N-acetylated oligosaccharide is selected from lacto-N-tetraose (LNT), lacto-N-neotetraose (LNnT) and combinations thereof.
40. Use according to any of paras 35 to 39, wherein 2′FL is administered to the subject in an amount of from about 0.5 g/day to about 5 g/day.
41. Use according to any of paras 35 to 40, wherein the one or more probiotic comprises or consists of Bifidobacterium infantis.
42. Use according to any of paras 35 to 41, wherein B. infantis is administered to the subject in a total amount of from about 10⁶ cfu/day to about 10¹² cfu/day.
43. Use according to any of paras 35 to 42, wherein the synbiotic comprises a probiotic mixture comprising B. infantis and at least one further probiotic, preferably wherein the at least one further probiotic comprises or consists of Bifidobacterium animalis and/or Lactobacillus rhamnosus.
44. The combination for use according to any of paras 13 to 18, wherein the composition comprises vitamin D in an amount of from about 5 μg/100 g to about 100 μg/100 g, on a dry weight basis.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A method of enhancing bone growth, strength and/or bone strength in a child or juvenile subject, the method comprising administering a combination of a vitamin mixture and a symbiotic to the child or juvenile subject, wherein the vitamin mixture comprises vitamin K2, vitamin A, and vitamin D, and wherein the synbiotic comprises 2′-fucosyllactose (2′FL) and one or more probiotic.

2. The method according to claim 1, wherein the vitamin K2 is administered to the child or juvenile subject in an amount from about 5 μg/day to about 200 μg/day; the vitamin A is administered to the child or juvenile subject in an amount from about 100 μg RE/day to about 1000 μg RE/day; and/or the vitamin D is administered to the child or juvenile subject in an amount from about 2.5 μg/day to about 100 μg/day.

3. The method according to claim 1, wherein the synbiotic comprises an oligosaccharide mixture comprising the 2′-fucosyllactose (2′FL) and at least one further oligosaccharide.

4. The method according to claim 3, wherein the at least one further oligosaccharide comprises one or more of:
(a) at least one sialylated oligosaccharide selected from the group consisting of 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof;
(b) at least one fucosylated oligosaccharide selected from the group consisting of 3-fucosyllactose (3FL), difucosyllactose (diFL), lacto-N-fucopentaose-I (LNFP-I), lacto-N-fucopentaose-II (LNFP-II), lacto-N-fucopentaose-III (LNFP-III), lacto-N-fucopentaose-V (LNFP-V), lacto-neofucopentaose V (LNnFP-V), lacto-N-difucosylhexaose-I (LNDFH-1), lacto-N-neodifucosylhexaose (LNnDFH), monofucosyllacto-n-hexaose-III (MFNLH-III), difucosyllacto-N-hexaose-a (DFLNHa) and combinations thereof; and/or
(c) at least one N-acetylated oligosaccharide selected from the group consisting of N-acetyl-glucosamine, N-acetyl-galactosamines, lacto-N-tetraose (LNT), lacto-N-neotetraose (LNnT), and combinations thereof.

5. The method according to claim 1, wherein the 2′FL is administered to the child or juvenile subject in an amount from about 0.5 g/day to about 5 g/day.

6. The method according to claim 1, wherein the one or more probiotic comprises Bifidobacterium infantis.

7. The method according to claim 1, wherein the one or more probiotic comprises a probiotic mixture comprising B. infantis and at least one further probiotic.

8. The method according to claim 1, wherein the combination is administered in a nutritional composition.

9. The method according to claim 1, wherein the child or juvenile subject is human.

10. The method according to claim 1, wherein the child or juvenile subject is an animal, wherein the animal is a pet.

11. The method according to claim 1, wherein the child or juvenile subject suffered from and/or is suffering from stunted growth and/or faltering growth.

12. The method according to claim 1, wherein the combination is administered by oral administration.

13. The method according to claim 1, wherein the combination is administered in an amount effective to promote osteoblast mineralization and/or osteoblast differentiation.

14. The method according to claim 1, wherein the combination is administered in an amount effective to improve one or more bone parameter selected from the group consisting of: bone mineral density (BMD), trabecular bone volume fraction (BV/TV), cortical bone volume (Ct.BV) and bone ultimate force (FMax).

15. The method according to claim 1, wherein the combination is administered in an amount effective to promote catch-up growth.

16. The method according to claim 1, wherein the child or juvenile subject is a human from 3 years to 10 years of age.

17. The method according to claim 1, wherein the combination is administered in a milk formula.

18. The method according to claim 3, wherein the at least one further oligosaccharide comprises one or more of:
(a) at least one sialylated oligosaccharide selected from the group consisting of 3′-sialyllactose (3′-SL), 6′-sialyllactose (6′-SL) and combinations thereof;
(b) at least one fucosylated oligosaccharide comprising difucosyllactose (diFL); and/or
(c) at least one N-acetylated oligosaccharide selected from the group consisting of lacto-N-tetraose (LNT), lacto-N-neotetraose (LNnT) and combinations thereof.

19. The method according to claim 6, wherein the B. infantis is administered to the child or juvenile subject in an amount from 10⁶ cfu/day to 10¹² cfu/day.


SEQUENCE LISTING (4 sequences)

SEQ ID NO: 1 - DNA, 21 bp, synthetic construct
  source 1..21: mol_type = other DNA
accatctttctgctcactctg

SEQ ID NO: 2 - DNA, 22 bp, synthetic construct
  source 1..22: mol_type = other DNA
gttcactaccttattgccctcc

SEQ ID NO: 3 - DNA, 20 bp, synthetic construct
  source 1..20: mol_type = other DNA
cactgaccggcctgtatgct

SEQ ID NO: 4 - DNA, 22 bp, synthetic construct
  source 1..22: mol_type = other DNA
gtatgttcggcttcccattctc